(19) United States
(12) Patent Application Publication
(10) Pub. No.: US 2010/0120682 A1
(43) Pub. Date: May 13, 2010

(54) COMPOSITIONS FOR INCREASING BODY WEIGHT, USE AND METHODS

(75) Inventors: Peter Arner, Bromma (SE); Göran Andersson, Huddinge (SE); Vanessa Van Harmelen, Stockholm (SE); Pernilla Lång, Holo (SE)

Correspondence Address: BROWDY AND NEIMARK, P.L.L.C.;624 NINTH STREET, NW, SUITE 300, WASHINGTON, DC, 20001-5303, US

(73) Assignee: Karolinska Innovations AB, Stockholm (SE)

(21) Appl. No.: 12/527,285

(22) PCT Filed: Feb. 13, 2008
(86) PCT No.: PCT/SE2008/050172

§ 371 Date: Oct. 14, 2009

Related U.S. Application Data
(60) Provisional application No. 60/889,820, filed on Feb. 14, 2007.

(30) Foreign Application Priority Data
Feb. 14, 2007 (SE) 0700363-5

(51) Int. Cl. A61K 38/17; A61P 3/00; A61K 38/22

(52) U.S. Cl. 514/12; 514/13

(57) ABSTRACT

Use of an isolated polypeptide comprising an amino acid sequence having a sequence identity of at least 80%, with the amino acid sequence represented by Gly Asn Ser Asp Asp Phe Leu Ser Gln Gln Pro Glu Arg Pro Arg AspVal Lys Leu Ala Arg (SEQ ID NO: 2), or a pharmaceutically acceptable salt thereof, for preparing a medicament for the treatment of a mammal to increase the body fat mass of said mammal, or to prevent or reduce the loss of body fat mass of said mammal. The polypeptide may comprise an amino acid sequence having a sequence identity of at least 80% with the amino acid sequence represented by SEQ ID NO: 1, and may be the polypeptide the monomeric proenzyme of tartrate-resistant acid phosphatase type 5 (TRAP). A pharmaceutical composition comprising the polypeptide. The polypeptide is useful for increasing the body fat mass of a mammal.

### FIELD OF THE INVENTION

The present invention relates to compounds useful for increasing the body weight of a mammal. More specifically, the invention relates to compounds useful for increasing the body fat mass of a mammal.

### BACKGROUND OF THE INVENTION

In the industrialized world, the number of persons suffering from an excess of body weight is steadily increasing. However, there also exist individuals that for one or another reason suffer from the opposite condition, viz. underweight. Underweight may be due to a subnormal lean body mass or a subnormal body fat mass, or a combination of both.

In an individual, the total body fat, or body fat mass, consists of essential fat and storage fat. Essential fat is stored in the marrow of bones, heart, lungs, liver, spleen, kidneys, intestines, muscles and lipid rich tissues of the central nervous system, and is necessary for normal physiological functioning. In women, the essential fat also is necessary for the reproductive function, and women carry more than four times as much essential fat as men.

The other type of body fat, storage fat, is fat accumulated in adipose tissue. Besides functioning as energy reserves in times of need, it has a function of e.g. protecting organs and generating body heat.

Underweight may be due to several causes, such as rapid metabolism, poor/inadequate diet or starvation (malnutrition), malabsorption due to defective intestinal function, endocrine disturbances e.g. type I diabetes, psychological problems (such as anorexia nervosa, body dysmorphic disorder, stress and anxiety) and weight loss, due to chronic illnesses and ageing. While in general the underlying cause of the underweight will have to be treated per se, the underweight too may be a health hazard, and as such have to be treated in itself. Indeed, persons suffering from underweight generally have poor physical stamina, a weakened immune system, as well as being at higher risk of developing diseases such as osteoporosis, heart disease and vascular disease. Additionally, in the female sex, underweight can lead to delayed sexual development, retarded amenorrhoea or complications during pregnancy.

The term cachexia is used for a condition of physical wasting with loss of body fat and muscle mass. Generally, cachexia may be associated with and due to conditions such as cancer, required immunodeficiency syndrome (AIDS), cardiac diseases, infectious diseases, shock, burn, endotoxinemia, organ inflammation, surgery, diabetes, collagen diseases, radiotherapy, and chemotherapy. In many of these diseases, cachexia may significantly contribute to morbidity or mortality.

Another particular group of individuals that are susceptible to developing a cachectic state are those individuals that have undergone a gastrectomy, such as may be practiced on gastric cancer and ulcer patients. Liedman et al., 1997 reported a loss of body weight of about 10% within the first six months after gastrectomic surgery, mainly due to loss of body fat, in patients having undergone either subtotal or total gastrectomy.

An estimated 0.5 to 3.7 percent of females in the US suffer from anorexia nervosa in their lifetime (American Psychiatric Association Work Group on Eating Disorders, 2000). According to data from the National Association of Anorexia Nervosa and Associated Disorders (US), the mortality rate of individuals suffering from anorexia is about 5% for each decade and increases up to 20% for patients that have the illness for more than 20 years. In the UK, the Eating Disorders Association has estimated an 18% mortality rate for anorexia nervosa. Anorexia nervosa mainly afflicts young people. A person developing anorexia nervosa before adulthood may suffer stunted growth and subsequent low levels of essential hormones (including sex hormones) as well as chronically increased cortisol levels.

From the above, it appears that while anorexia nervosa admittedly may have psychological and socio-cultural causes, the condition, if permitted to develop too far, may need urgent clinical treatment including hospitalization and intravenous infusion or nasogastric tube-feeding of nutritients.

Attempts have been made to provide means of treating conditions of underweight or reduced body fat, such as cachexia and anorexia. Thus, U.S. Pat. No. 7,015,241 describes a method of treating anorexia by use of a sulfonamide derivative or sulfonic acid ester derivative that is said to stimulate the appetite. U.S. Pat. No. 6,387,883 describes methods of inhibiting metabolic and cytokine associated features of cachexia by use of a nutritional composition comprising an effective amount of various omega-3 fatty acids. U.S. Pat. No. 7,138,372 discloses an agent for preventing and/or treating cachexia comprising Tumour Cytotoxic Factor-II (TCF-II) or hepatocyte growth factor (HGF) as an effective ingredient.

WO04032952A1 describes the use of ghrelin or an analogue thereof for the preparation of a medicament for one or more of: treatment and/or prevention of loss of body weight and body fat, prophylaxis or treatment of cachexia, stimulation of appetite, stimulation of food intake, stimulation of weight gain, or increasing body fat mass, in a gastrectomized individual.

In cancer patients, in particular, various agents have been administered in attempts to retard or halt progressive cachexia. Thus, it has been suggested to use corticosteroids to alleviate symptoms such as anorexia, asthenia, and pain in patients with cancer. Significant improvements in appetite and a sense of well-being have been reported in randomized trials with prednisolone, methylprednisolone, or dexamethasone, though these improvements were not long-lasting and did not remain after completion of the studies (Willox et al., 1984; Bruera et al., 1985; Popiela et al., 1989; Della Cuna et al., 1989).

Megestrol acetate also has been observed to stimulate appetite and produce weight gain in a variety of cachectic cancer patients (Maltoni et al., 2001). In a study of the combination of appetite-stimulating properties of megestrol acetate and the antiinflammatory properties of ibuprofen it was suggested that this combination stabilized quality of life and produced weight gain in patients with advanced gastrointestinal cancer (McMillan et al., 1999).

Medroxyprogesterone acetate, a synthetic progestogen, also has been shown to increase appetite, however, without any weight gain being observed (Downer et al., 1993; Simons et al., 1996).

The cannabinoid dronabinol is another compound that has been found to stimulate appetite. In a study on cancer patients, higher doses of the compound, of 5.0 or 7.5 mg/d, were found to be more effective than the low dose of 2.5 mg/d, though, nonetheless, the patients continued to lose weight (Plasse et al., 1991; Nelson et al., 1994).

Another compound, the serotonin antagonist cyproheptadine, has been observed to produce weight gain in clinical situations. However, in patients with advanced malignant neoplasms, cyproheptadine treatment, while resulting in a decrease in nausea and mild enhancement in appetite, did not abate progressive weight loss (Kardinal et al., 1990).

The prokinetic agent Metoclopramide, at a dosage of 10 mg orally 4 times daily before meals and at bedtime, was shown to be effective in stimulating appetite and relieving other dyspeptic symptoms associated with anorexia in advanced cancer patients with delayed gastric emptying or gastroparesis (Nelson et al., 1993; Shivshanker et al., 1983).

Eicosapentaenoic acid (EPA), an ω-3 polyunsaturated fatty acid, has been shown to possess antitumor, as well as anticachexia, activities in animal cachexia models, inducing inhibition of weight loss accompanied by increases in total body fat and muscle mass (Dagnelie et al., 1994; Tisdale et al., 1990). In one study it was shown that while nutritional supplement alone did not attenuate the development of weight loss in cachectic cancer patients, nutritional supplement enriched with EPA resulted in significant weight gain (Barber et al., 1999).

The growth hormone secretagogue ghrelin has been shown to be capable of stimulating adiposity. Also, subcutaneous injections of a more stable synthetic ghrelin-receptor agonist GHRP-2 (growth hormone releasing peptide-2) were observed to produce dose-dependent increases in food intake and body weight (Tschop et al., 2002).

The inhibitor of prostaglandin synthesis ibuprofen has been reported to produce body weight gain, and to improve survival in cachectic cancer patients (Preston et al., 1995; Wigmore et al., 1995; Lundholm et al., 1994).

Finally, the antidiabetic drug glitazone (a thiazolidinedione) increases body weight considerably, which at least in part is due to increase in fat mass (Stumvoll & Haring, 2002; Fonesca, 2003). The effect is mediated by activation of the specific fat cell receptor PPAR-gamma, which, in turn, stimulates formation of new fat cells and also enhances lipid storage in adipocytes.

The metallo-enzyme tartrate resistant acid phosphatase (TRAP), also known as uteroferrin, purple acid phosphatase or type 5 acid phosphatase (Acp5), is a basic, iron-binding protein in mammals with high activity towards e.g. phosphoproteins and ATP. It exists either as a latent monomeric pro-enzyme of approximately 35 kDa or as a proteolytically processed two-subunit enzyme of approximately 22 and 16 kDa, respectively, linked by a disulphide bridge (Lang and Andersson, 2005; Ljusberg et al., 1999). The proteolytic processing (exerted by, for instance, cathepsin K) of the monomeric proenzyme excises part of an exposed loop region close to the active site and is permissive for the catalytic activation of TRAP (Ljusberg et al., 2005).

TRAP is found in a variety of organs, such as bone, spleen, lung and placenta and is found in various cell types. It is secreted in vivo by osteoclasts (Hollberg et al., 2005; Reinholt et al., 1990) and has been shown to be secreted in vitro by both macrophages and osteoclasts (Janckila et al., 2005). Intracellularly, TRAP is thought to participate in degradation of collagen fragments as well as of phagocytosed bacteria in osteoclasts (Halleen et al., 1999) and macrophages (Räisanen et al, 2005), respectively. Osteoclast secreted extracellular TRAP, on the other hand, has been proposed to participate in the regulation of osteoclast adhesion and migration (Andersson et al., 2003). However, the role of extracellular TRAP secreted from other cell types, including macrophages, is unknown.

The amino acid sequence of TRAP is represented by the sequence

(SEQ ID NO: 1)

Met Asp Met Trp Thr Ala Leu Leu Ile Leu Gln Ala
Leu Leu Leu Pro Ser Leu Ala Asp Gly Ala Thr Pro
Ala Leu Arg Phe Val Ala Val Gly Asp Trp Gly Gly
Val Pro Asn Ala Pro Phe His Thr Ala Arg Glu Met
Ala Asn Ala Lys Glu Ile Ala Arg Thr Val Gln Ile
Leu Gly Ala Asp Phe Ile Leu Ser Leu Gly Asp Asn
Phe Tyr Phe Thr Gly Val Gln Asp Ile Asn Asp Lys
Arg Phe Gln Glu Thr Phe Glu Asp Val Phe Ser Asp
Arg Ser Leu Arg Lys Val Pro Trp Tyr Val Leu Ala
Gly Asn His Asp His Leu Gly Asn Val Ser Ala Gln
Ile Ala Tyr Ser Lys Ile Ser Lys Arg Trp Asn Phe
Pro Ser Pro Phe Tyr Arg Leu His Phe Lys Ile Pro
Gln Thr Asn Val Ser Val Ala Ile Phe Met Leu Asp
Thr Val Thr Leu Cys Gly Asn Ser Asp Asp Phe Leu
Ser Gln Gln Pro Glu Arg Pro Arg Asp Val Lys Leu
Ala Arg Thr Gln Leu Ser Trp Leu Lys Lys Gln Leu
Ala Ala Ala Arg Glu Asp Tyr Val Leu Val Ala Gly
His Tyr Pro Val Trp Ser Ile Ala Glu His Gly Pro
Thr His Cys Leu Val Lys Gln Leu Arg Pro Leu Leu
Ala Thr Tyr Gly Val Thr Ala Tyr Leu Cys Gly His
Asp His Asn Leu Gln Tyr Leu Gln Asp Glu Asn Gly
Val Gly Tyr Val Leu Ser Gly Ala Gly Asn Phe Met
Asp Pro Ser Lys Arg His Gln Arg Lys Val Pro Asn
Gly Tyr Leu Arg Phe His Tyr Gly Thr Glu Asp Ser
Leu Gly Gly Phe Ala Tyr Val Glu Ile Ser Ser Lys
Glu Met Thr Val Thr Tyr Ile Glu Ala Ser Gly Lys
Ser Leu Phe Lys Thr Arg Leu Pro Arg Arg Ala Arg
Pro.

In this amino acid sequence, the exposed loop region, the excision of which results in the catalytic activation of the enzyme, is represented by:

(SEQ ID NO: 2)

Gly Asn Ser Asp Asp Phe Leu Ser Gln Gln Pro Glu
Arg Pro Arg Asp Val Lys Leu Ala Arg,

and corresponds to the sequence of the monomeric TRAP proenzyme located from amino acid no. 162 to amino acid no. 182.

The use of TRAP in diagnosis is disclosed in US patent application No. 20050074800, wherein methods of diagnosing joint disease are provided, comprising measuring concentration or activity of at least one joint disease-diagnostic enzyme, e.g. TRAP, in a tissue, cell, or fluid test sample taken from a joint of a test subject.

US patent application No. 20040228899, on the other hand, discloses devices suitable for orthopedic or dental implantation to bone, having TRAP adsorbed to a porous hydroxyapatite substratum.

In both cited US application, it is the involvement of TRAP in bone metabolism that is exploited.

PCT publication WO04041170 relates to relates to compositions containing proteins and methods of using those compositions for the diagnosis and treatment of immune related diseases. In the Sequence listing, containing more than 2500 sequences, the polypeptide sequence of TRAP is present. However, there is no example of use of this polypeptide in the description, which is largely speculative on the possible therapeutic utility of the polypeptides.

### SUMMARY OF THE INVENTION

From the above description, it appears that there still exists a need for efficacious drugs for treating or preventing a condition of subnormal body fat mass, such as may be found in a cancer or heart failure patient, in an individual suffering from cachexia or in a person suffering from an eating disorder and in elderly patients who cannot achieve adequate nutrition. One object of the present invention is to provide such a drug.

This object is achieved, according to the present invention, on the basis of the surprising discovery, made by the present inventors, of a hitherto unknown and completely unexpected effect of the monomeric TRAP proenzyme on adipogenesis.

Thus, according to one aspect the present invention provides the use of an isolated polypeptide comprising an amino acid sequence having a sequence identity of at least 80%, preferably at least 85%, more preferably at least 90%, most preferably at least 95%, with the amino acid sequence represented by SEQ ID NO: 2, or a pharmaceutically acceptable salt thereof, for preparing a medicament for the treatment of a mammal to increase the body fat mass of said mammal, or to prevent or reduce loss of body fat mass of said mammal.

According to one aspect, the present invention provides an isolated polypeptide comprising an amino acid sequence having a sequence identity of at least 80%, preferably at least 85%, more preferably at least 90%, most preferably at least 95%, with the amino acid sequence represented by SEQ ID NO: 2, or a pharmaceutically acceptable salt thereof, for use as a medicament.

According to another aspect, the present invention provides a pharmaceutical composition comprising an isolated polypeptide comprising an amino acid sequence having a sequence identity of at least 80%, preferably at least 85%, more preferably at least 90%, most preferably at least 95%, with the amino acid sequence represented by SEQ ID NO: 2, or a pharmaceutically acceptable salt thereof, as well as a method of treatment of a mammal by administering the pharmaceutical composition to said mammal.

According to still another aspect, the present invention provides an isolated polypeptide comprising an amino acid sequence represented by SEQ ID NO: 2 or an amino acid sequence having a sequence identity with the amino acid sequence represented by SEQ ID NO: 2 of at least 80%, preferably at least 85%, more preferably at least 90%, most preferably at least 95%, for use as a medicament.

In one embodiment of the invention, the polypeptide comprises an amino acid sequence having a sequence identity of at least 80%, or at least 90%, preferably at least 95%, more preferably at least 98%, most preferably at least 99%, with the amino acid sequence represented by SEQ ID NO: 1.

In this embodiment, thus, the polypeptide comprises an amino acid sequence having a sequence identity of at least at least at least 80%, or at least 90%, preferably at least 95%, more preferably at least 98%, most preferably at least 99%, with the amino acid sequence represented by SEQ ID NO: 1, which amino acid sequence comprises an amino acid sequence having a sequence identity of at least 80%, preferably at least 85%, more preferably at least 90%, most preferably at least 95%, with the amino acid sequence represented by SEQ ID NO: 2.

In another embodiment of the invention, the polypeptide comprises monomeric proenzyme of TRAP.

According to another aspect, the invention provides the use of a compound capable of reducing the proteolytic processing of monomeric pro-enzyme of TRAP, for preparing a medicament for the treatment of a mammal to increase the body fat mass of said mammal, or to prevent or reduce the loss of body fat mass of said mammal.

Further aspects and embodiments of the invention are as defined in the claims.

### BRIEF DESCRIPTION OF THE DRAWINGS

FIG. 1. Characterization of adipose tissue from WT and TRAP+ mice. (A) TRAP+ mice are significantly heavier than WT at all ages (wt n>6, TRAP+n>4 at all time points). (B) TRAP+ mice have significantly higher content of total body fat, but have no sign of apparent overeating. (C) Gpd2 is significantly increased in TRAP+ mice compared to WT mice and PPARγ and show tendency to increase, while pref-1 shows a tendency of decrease. (D) No difference in adipocyte volume between WT and TRAP+ mice is noted.

FIG. 2. Characterization of adipose tissue from WT and TRAP+p mice. (A) Growth curve for TRAP+p mice compared to WT mice. No significant difference in body weight was found at any time point (wt n=6, TRAP+n=3 at all time points). (B) Total body fat in TRAP+p vs. WT mice. No significant difference in body fat content was found. (C) TRAP mRNA and TRAP enzyme activity in adipose tissue of TRAP+p compared to WT. (D) Expression of monomeric and proteolytically processed TRAP protein in adipose tissue of WT, TRAP+p and TRAP+ mice. Equal loading of enzyme activity shows that TRAP+ (lane 3) express more monomeric TRAP compared to TRAP+p (lane 2). (E) Neither PPAR9, Gpd2 or pref-1 are significantly changed compared to WT.

p<0.05; **p<0.01; ***p<0.005

FIG. 3. Expression of TRAP mRNA and monomeric TRAP protein in human obesity. Expression of TRAP mRNA and monomeric TRAP protein is increased in obesity compared to the lean state (A). TRAP mRNA in further increased in patients displaying a more hyperplastic phenotype than hypertrophic phenotype (B). Monomeric TRAP protein is further increased in patients displaying a more hyperplastic phenotype than hypertrophic phenotype (C).

FIG. 4. Stimulation of adipocyte proliferation and differentiation by monomeric TRAP (TRAP mono) but not cleaved TRAP (TRAP cl). The following cells were used: Mouse 3T3-L1 preadipocytes (A, B), Human mesenchymal stem cells (hMSC) (C, D) and human pre-adipocytes (E, F). hMSC were treated with TRAP either before (Pro) or at (Post) confluence. Data are expressed as percentage of control. *p<0.05.

FIG. 5. Macrophages secrete and are the primary source of monomeric TRAP in mouse and human adipose tissue. (A) mRNA for the monocyte/macrophage marker F4/80 and c-fms show a tendency towards or are significantly increased in adipose tissue of TRAP+ vs. WT mice. (B) Increased labelling for mouse macrophage marker F4/80 in adipose tissue from TRAP+ compared to WT mice (C) mRNA for the myeloid lineage specific TRAP transcript 1C is predominant in adipose tissue of TRAP+ and WT mice. (D) TRAP mRNA expression is significantly higher in human adipose tissue than in the adipocytes fraction. (E) In five subjects (Pat 1-5) monomeric TRAP is more abundantly expressed in human adipose tissue than in isolated adipocytes (Ad). (F) Co-localization between monomeric or total TRAP and the macrophage marker CD68 in subcutaneous human adipose tissue from a representative subject. (G) Monomeric TRAP is secreted from the mouse macrophage cell line RAW 264.7 (lane 1), whereas proteolytically processed TRAP is not detectable (lane 2).

FIG. 6. Metabolic and inflammatory profile of adipose tissue from obese TRAP overexpressing mice. (A) Levels of leptin and adiponectin mRNA and serum protein in WT and TRAP+ mice. TRAP+ has two fold higher level of serum leptin than the WT mice, due to increased body weight. No significant change was found in the mRNA or serum levels for adiponektin. (B) Inflammatory profile in the TRAP+ mice. TRAP+ mice have increased expression of TNFα mRNA. Other cytokines, such as ILL IL6 and CCL2, are unchanged between the TRAP+ and WT mice. (C) Noradrenaline stimulated lipolysis in isolated adipocytes from TRAP+ and WT mice. There was no difference in the noradrenaline stimulated lipolysis between TRAP+ and WT mice. (D) Insulin inhibited lipolysis in isolated adipocytes from TRAP+ and WT mice. There was no difference in insulin inhibited lipolysis between TRAP+ and WT mice. (E) Insulin stimulated lipogenes in isolated adipocytes from TRAP+ and WT mice. There was no difference in insulin stimulated lipogenes between TRAP+ and WT mice. (E) Level of blood glucose and insulin in TRAP+ mice. No difference was seen in glucose, insulin and HOMA-index between TRAP+ and WT mice.

### DETAILED DESCRIPTION OF THE INVENTION

For the purpose of the present invention the term polypeptide is used analogously with oligopeptide, peptide and protein, if nothing else is indicated or apparent from the context.

The term “amino acid” should be construed as comprising both natural and non-natural amino acids. Any optically active amino acid may be in the “D” or “L” isomeric form.

For the purpose of the present invention, the term “identity” is used analogously with the term “homology”, referring to the percentage of amino acid residues in the a given amino acid sequence that are identical with the residue of the sequence to which it is compared. Thus, as an example, two polypeptides that are 90% homologous have a 90% sequence identity.

To assess identity between any two sequences, the sequences are aligned and, if necessary, gaps are introduced. The skilled person will be well aware of methods for performing such alignments, using any of the sequence analysis software packages that are commercially available.

Homologous polypeptides may comprise one or more, conservative or non-conservative, preferably conservative, amino acid substitutions. In a conservative amino acid substitution the substituting amino acid has similar size, hydrophobicity and hydrophilicity values, as well as similar electronic properties as the amino acid being substituted. As an example, substituting an alanine residue for a valine residue is considered a conservative substitution of one non-polar amino acid for another, while substituting a glutamic acid residue for an aspartic acid residue is considered a conservative substitution of one acidic amino acid for another, and so on. It is well within the knowledge of the skilled person to appreciate what amino acid may replace another one in order to obtain a conservative substitution.

A homologue of a polypeptide also may be one wherein at least one amino acid has been inserted or deleted, typically from 1 to 5 amino acids. To determine which amino acid residues can be substituted, inserted, or deleted while maintaining the enzymatic activity computer programs well known to the skilled person may be used, e.g. the DNAstar software (www.dnastar.com).

Any amino acid as well as the C-terminus and/or the N-terminus of the polypeptide may also be substituted by a moiety that may provide the polypeptide with some other beneficial feature, such as improved solubility, absorption and/or biological half life, which moiety may also be a protecting group of any functional group of the polypeptide. Suitable protecting groups are described in Greene's Protective Groups in Organic Synthesis, Peter G. M. Wuts, Theodora W. Greene, fourth ed., 2006, ISBN: 0471697540.

Examples of N-terminal protecting groups include acyl groups, while examples of C-terminal protecting groups include amine groups.

It will be well within the knowledge of the skilled person to identify suitable homologues and derivatives, e.g. having suitable N and/or C terminal protecting groups, of the inventive polypeptides, by preparing and assaying the homologue for adipogenetic effect, e.g. in an in vitro system, e.g. applying assay methods as described herein below.

The polypeptides of the present invention may be prepared by recombinant DNA techniques in cellular systems, e.g. microorganisms, such as bacteria and yeasts, or in insect or vertebrate cells. These techniques are well-known to the person skilled in the art, and suitable protocols are described e.g. in Sambrook, J.; Russell, D.; Molecular Cloning; A Laboratory Manual Cold Spring Habor, third edition, 2001.

In addition, the polypeptides of the invention can be chemically synthesized, cf. e.g., Creighton, T. E., Proteins: Structures and Molecular Principles, second edition, 1993, ISBN: 0716723174.

Non-classical amino acids or chemical amino acid analogs can be incorporated into the polypeptide by substitution or addition. Examples of non-classical amino acids are citrulline, homocitrulline, hydroxyproline, norleucine, norvaline, ornithine, sarcosine etc.

According to one aspect of the present invention, a pharmaceutical composition is provided, comprising an isolated polypeptide comprising an amino acid sequence having a sequence identity of at least 80%, preferably at least 85%, more preferably at least 90%, most preferably at least 95%, with the amino acid sequence represented by SEQ ID NO: 2, and/or an isolated polypeptide comprising an amino acid sequence having a sequence identity of at least 80%, or at least 90%, preferably at least 95%, more preferably at least 98%, most preferably at least 99%, with the amino acid sequence represented by SEQ ID NO: 1 and/or the monomeric pro-enzyme of tartrate-resistant acid phosphatase type 5 (TRAP).

The pharmaceutical composition of the invention preferably is in a form suitable for injection, e.g. subcutaneous injection, or in a form suitable for intravenous infusion. It may include a solution or dispersion of a polypeptide of the invention in a suitable, pharmaceutically acceptable carrier, for example, water, ethanol, polyol and suitable mixtures thereof, and vegetable oils.

The pharmaceutical composition also may be provided as a sterile powder for the extemporaneous preparation of a sterile solution or dispersion for injection.

The pharmaceutical composition of the invention should be stable under the conditions of manufacture and storage. Furthermore, it should be preserved against the contaminating action of microorganisms such as bacteria and fungi. To this end, an antibacterial agent and/or antifungal agent may be added, e.g. chlorobutanol, sorbic acid, thimerosal, and the like.

The pharmaceutical composition additionally may contain an isotonic agent, such as a sugar or sodium chloride. Also, a composition having a prolonged absorption can be prepared by the use of agents delaying absorption, for example, aluminum monostearate and gelatin.

As is well-known to the person skilled in the art, a sterile solution for injection may be prepared by admixing the active ingredient in a suitable solvent, optionally with any additive, e.g. such as mentioned herein above, and filtered sterilizing the solution thus obtained. A dispersion for injection may be prepared by admixing the active ingredient in a sterile carrier comprising the basic dispersion medium and optionally any additive, e.g. such as mentioned herein above.

The present invention also includes pharmaceutically acceptable salts of the polypeptides of the invention. Any such salt, of course, must have essentially the required biological activity according to the invention.

Examples of pharmaceutically acceptable salts are acid addition salts such as salts with hydrochloric, phosphoric, sulphuric acids, and organic acids, such as tartaric, acetic, citric, malic and lactic, fumaric acids; and pharmaceutically acceptable salts with alkali metals or alkaline earth metals or lower alkylammonium salts.

The medicament of the invention will comprise a therapeutically effective amount of the polypeptide of the invention, together with pharmaceutically acceptable carrier(s) and excipient(s), so as to provide a therapeutically effective dose for increasing the body fat mass of a mammal on administration thereof to the mammal, preventing a decrease of the body fat mass of the mammal, or arresting or reversing a decrease of the body fat mass of the mammal. The skilled person will be aware of methods for determining a therapeutically effective dose, by standard preclinical and clinical test procedures using cell cultures and/or experimental animals.

In any particular case, the administered dose may be determined by a physician, taking account of parameters such as the age, weight, condition and the type and severity of the disease of the patient to be treated. Typically, a therapeutically effective amount of the peptide or peptide derivative can range from about 0.1 mg per day to about 1000 mg per day for an adult. A typical daily dose is from about 0.1 to 50 mg per kg, preferably from about 1.0 mg per kg to 10 mg per kg of body weight, according to the activity of the specific therapeutic substance, the age, weight and condition of the subject to be treated, and the frequency and route of administration.

The polypeptide of the invention may be administered with at least one biologically active substance, either simultaneously or sequentially. If administered simultaneously, the polypeptide and the other biologically active substance(s) may be provided in separate compositions or combined within the same composition.

Examples of biologically active substances that may be used according to the present invention are compounds capable of increasing the general well-being and/or stimulating appetite and/or increasing the body weight; and compounds capable of reducing the proteolytic processing of monomeric pro-enzyme TRAP and/or compounds capable of reducing the proteolytic processing of the polypeptide of the invention.

Examples of compounds capable of increasing the general well-being and/or stimulating appetite and/or increasing the body weight are mentioned herein above in relation to the prior art, and are e.g. omega-3 fatty acids such as eicosapentaenoic acid; corticosteroids such as methylprednisolone, dexamethasone and prednisolone; megestrol acetate; medroxyprogesterone acetate; dronabinol; cyproheptadine; metoclopramide and ibuprofen, and it is contemplated that the biologically active agent may be selected from any of these, as well as from other compounds of similar effects as known to the skilled person, e.g. as mentioned in some of the patent documents referred to herein above.

Furthermore, it is contemplated that the polypeptide of the invention may be administered in combination with several of the above-mentioned other biologically active substances and/or in combination with one or several other biologically, optionally therapeutically, active substances as may be indicated in view of any particular condition associated with the subnormal body fat mass.

In one embodiment, it is contemplated that the polypeptide of the invention, optionally in combination with any of the above-mentioned other active substances, is administered in combination with a dosage of lipids or a diet enriched in nutritionally assimilable lipids.

Compounds capable of reducing the proteolytic processing of monomeric pro-enzyme TRAP are e.g. inhibitors of cathepsin K. Examples of such inhibitors are given e.g. in U.S. Pat. Nos. 6,274,336, 7,012,075, and in US patent application No. 20060122268.

As indicated herein above, the body fat mass can be measured by number of methods, and the term “body fat mass”, as used herein, should be construed as measured by any of these methods, known to the person skilled in the art.

The simplest way of determining a possible change of body fat mass, e.g. a loss of body fat mass, of an individual is by weighing the individual. Though of course this method does not take account of the possible change of body weight due to e.g. a change in muscle mass, it nonetheless may give a first indication of any change of the body fat mass. Also, such measurement may serve as a basis for determining the body mass index (BMI) of the individual. The BMI, defined as the body weight of an individual divided by the square of his or hers height, provides a simple means of assessing how much an individual's body weight departs from what is normal or desirable for a person of his or her height. Common definitions of BMI categories are as follows: starvation: BMI—less than 15 kg/m²; underweight—BMI less than 18.5 kg/m²; ideal—BMI from 18.5 to 25 kg/m²; overweight—BMI from 25 to 30 kg/m²; obese—BMI from 30 to 40 kg/m²; morbidly obese—BMI greater than 40 kg/m². In general, people suffering from anorexia nervosa have a BMI of less than 17.5 kg/m².

While simple, the BMI method of characterizing the body weight property of a person is not always correct. For example, the BMI does not take into account factors such as frame size, muscularity or varying proportions of e.g. bone, cartilage, and water weight among individuals. Thus, the accuracy of BMI in relation to actual levels of body fat mass may be distorted by such factors as fitness level, muscle mass, bone structure, gender, and ethnicity. Also, people with short stature and old people tend to have lower BMI values. It is considered, however, that the skilled person, e.g. a physician, will be able to take these factors into account when making the BMI assessment of any given individual.

Nevertheless, BMI categories are generally regarded as a satisfactory tool for measuring whether sedentary individuals are e.g. “underweight,” “overweight” or “obese”.

In one embodiment of the invention, the medicament is for treating a mammal suffering from a condition associated with a subnormal BMI or with a susceptibility of developing a subnormal BMI.

In one embodiment, a BMI lower than 18.5, e.g. lower than 18, lower than 17.5 or even lower than 17 is considered subnormal.

The body fat percentage provides another means of characterizing the body of an individual. The body fat percentage is the fraction of the total body mass that is adipose tissue, or body fat mass, the rest being the so-called lean body mass, e.g. bone, muscle, organ tissue, etc. In the US, the National Institute of Health has indicated that a recommended body fat percentage for women is 20-21%, and that for men it is 13-17%.

In one embodiment of the invention, the medicament is for the treatment of a mammal suffering from a condition associated with a subnormal body fat mass or body fat percentage or with a susceptibility of developing a subnormal body fat mass or body fat percentage.

In one embodiment, in a woman, a body fat percentage of lower than 20%, or a body fat percentage of lower than 19%, e.g. a body fat percentage of lower than 18% is considered as a subnormal value, whereas in a man, a body fat percentage of lower than 13%, or a body fat percentage of lower than 12%, e.g. a body fat percentage of lower than 11% is considered a subnormal value.

There exist several methods of determining the total body fat mass, well-known to the person skilled in the art, e.g. by Dual energy X-ray Absorptiometry (DXA); Average Density Measurement (Hydrostatic Weighing), or Bioelectrical Impedance Analysis (BIA); or by the simpler skinfold test (Durnin J. V. G. A. and M. M. Rahaman, 1967). In this latter test, the skinfold thickness is measured at various, representative sites on the body by use of calipers, and the measured data are used to calculate body fat by different calculation methods. As an example, Harpenden Skinfold Calipers are commercially available with detailed instructions manual, cf. www.fitnessassist.co.uk

For the purpose of the present invention, the terms “mammal” and “individual” are intended to refer to either an animal or human individual, if the contrary is not indicated or obvious from the context. The animal may be e.g. a farm animal, a domestic animal, a laboratory animal or a pet animal, e.g. a dog, a cat, a pig, a cow, a sheep, a rat, a rabbit, a mouse etc. Preferably, the mammal/individual is a human.

All of the above prior art documents referred to herein above are incorporated by reference.

Herein below, the invention will be illustrated by the following non-limiting examples.

### EXAMPLES

### Material and Methods

### Experimental Animals

Age-matched male and female mice from different litters (WT, TRAP+p and TRAP+) were kept under controlled light/dark conditions with food and water available ad libitum.

### Generation and Genotyping of TRAP Overexpressing Transgenic FVB/N Mice

TRAP overexpressing transgenic FVB/N mice (FVB/N-trap+ or FVB/N-trap+p) were generated as previously described (Angel et al., 2000) and each litter was genotyped. Transgenic animals containing >30 copies of the TRAP gene were used.

Genomic DNA was purified using Puregene (Gentra, Minneapolis, Minn.) according to the manufacturer's protocol for DNA purification from mouse-tail. Primers (Invitrogen, Carlsbad, Calif.)/probes (Biosearch Technologies, Novato, Calif.) for SV40 and TRAP were used. The TRAP primers and probes were as follows: 5′ GCTACTTGCGGTTTCACTATGGA 3′ (SEQ ID NO: 3) and 5′ TGGTCATTTCTTTGGGGCTTATCT 3′ (SEQ ID NO: 4), and FAM labeled probe 5′TGTGAAGCCGCCCAGGGAGTCCTC 3′ (SEQ ID NO: 5), annealing temperature 62° C. qPCR was run as stated under “Total RNA purification and RT-qPCR” with the exception that iQ Supermix (Bio-Rad, Hercules, Calif.) was used.

### Measurement of Lean Mass and Body Fat Content Using DXA

Dual-energy X-ray absorptiometry (DXA) was performed as described [27]. Male (WT; n=24, TRAP+p; n=17, TRAP+; n=5) and female (WT; n=28, TRAP+p; n=14, TRAP+; n=9), 2-12 month mice were used (Supplementary Table IV). Statistical analysis was carried out using Kruskal Wallis test followed by Mann-Whitney U test.

### DXA Measurement of Body Fat Content

Dual-energy X-ray absorptiometry was performed as described elsewhere (Nagy and Clair, 2000). Male (WT; n=24, TRAP+p; n=17, TRAP+; n=5) and female (WT; n=28, TRAP+p; n=14, TRAP+; n=9), 2-12 month mice were used. Statistical analysis was carried out using Kruskal Wallis test followed by Mann-Whitney U test.

### Calculation of Adipocyte Volume

Adipocyte size was determined as described (Reynisdottir et al., 1994). Adipose tissue was obtained from the animals (WT; n=8, TRAP+; n=8) and adipocytes were isolated by collagenase treatment. Using direct microscopy, the diameter of 100 cells was determined and the mean fat cell volume was calculated. Statistical analysis was carried out using ANOVA.

### Measurement of Food Intake

Animals were given a specific amount of food and the consumption was measured by weighing the remaining food every other day for two weeks. Food consumption was then calculated as intake of gram food/gram body weight over two weeks for male (WT; n=3, TRAP+; n=6) and female (WT; n=3, TRAP+; n=5) mice. Statistical analysis was carried out using Mann-Whitney U test.

### Total RNA Purification and RT-qPCR on Mouse and Human Tissue

Total RNA was extracted from gonadal or mesenteric adipose tissue of male and female mice (WT; n=11, TRAP+p; n=7, TRAP+; n=9) using RNeasy Lipid Tissue Mini Kit (QIAGEN, Hilden, Germany), treated with DNase (Invitrogen, Carlsbad, Calif.), quantified using Ribogreen (Invitrogen, Carlsbad, Calif.) and then transcribed using iScript cDNA Synthesis Kit (Bio-Rad, Hercules, Calif.). From human subjects, total RNA was extracted from 300 mg subcutaneous fat tissue (n=28) using RNeasy Mini Kit (QIAGEN, Hilden, Germany), determination of RNA purity and reverse transcription was performed as previously described (Hoffstedt et al., 2004). Real-Time PCR was carried out on an iCycler iQ Real Time PCR Detection System (Bio-Rad, Hercules, Calif.) in triplets using, for mouse cDNA, Platinum® SYBR® Green qPCR SuperMix UDG (Invitrogen, Carlsbad, Calif.) and, for human cDNA, iQ SYBR Green Supermix (Bio-Rad, Hercules, Calif.) both with the addition of 10 nM fluorescein (Bio-Rad, Hercules, Calif.) in a final volume of 25 μl. Program setting for mouse samples were as follows: 2 min at 50° C., 2 min at 95° C., 40 cycles of 15 sec at 95° C. and 30 sec at 62-64° C. (depending on primer pair). Program setting for human samples were as follows: 10 min at 95° C., 40 cycles of 15 sec at 95° C. 20 sec at 63° C. Primer pairs were optimized prior to use for primer concentration and annealing temperature to achieve PCR amplification efficiency between 95-105%. Mouse samples were normalized towards β-actin and human samples towards GAPDH. Relative quantification of mRNA was calculated using the “Comparative Ct method” as described in User Bulletin 2 from Applied Biosystems. For mice, statistical analysis was carried out using Mann-Whitney U test and for humans using ANOVA. Information in respect of the TRAP qPCR primers and conditions of use is given in Table 1, wherein Ann. temp. stands for the annealing temperature.

TABLE 1
TRAP Primer information for qPCR

| | | | SEQ | Primer conc. | Ann. temp. |
|---|---|---|---|---|---|
| Gene/mRNA transcript | Species | Nucleotide sequence | ID NO: | (nM) | (° C.) |
| TRAP | human | CGCACAGGTAGGCAGTGAC* | 6 | 300 | 63 |
| | | CTACCCCGTGTGGTCCATAG** | 7 | 300 | |
| TRAP | mouse | GCTACTTGCGGTTTCACTATGGA* | 3 | 900 | 62 |
| | | TGGTCATTTCTTTGGGGCTTATCT** | 4 | 300 | |
| TRAP 1A transcript | mouse | GGTCAGGAGTGGGAGCCATAT* | 8 | 900 | 60 |
| | | AAGAGCCTTCAAGTAAGTGGAACA** | 9 | 900 | |
| TRAP 1B transcript | mouse | TCCGCAGCTCAGTTGGGTAG* | 10 | 300 | 59 |
| | | GCCCACAGCCACAAATCTCAG** | 11 | 300 | |
| TRAP 1C transcript | mouse | CTCTGACCACCTGTGCTTCCT* | 12 | 900 | 65 |
| | | CTGTGTGGAATGGGGCATTGG** | 13 | 900 | |

*Sense primer;
**Antisense primer

Measurement of TRAP Enzyme Activity Using pNPP as Substrate

TRAP enzyme activity was measured (Lang et al., 2001) in tissue homogenates prepared as follows. Adipose tissue from male and female mice (WT; n=11, TRAP+; n=10, TRAP+p; n=10) was homogenized in 0.15 M KCl+0.1% Triton X-100+Pefabloc (10 mg/ml)+Complete, Protease Inhibitor Cocktail Tablets (1 tablet/50 ml solution) (Boehringer Mannheim, Mannheim, Germany) and centrifuged at 3200×g for 30 minutes. The supernatant was then assayed for TRAP enzyme activity.

TRAP Protein is Effectively Taken Up by the Cells after In Vivo Administration to Rats

Recombinant monomeric TRAP (5 μg in 100 μl) was administered to 6 adult rats in vivo by local infusion into gonadal fat pads using osmotic pumps for 7 days. The contralateral fat pad in the same animal received the same treatment except that recombinant TRAP was omitted and instead only vehicle was infused for the same time period. The area of adipose tissue close to the site infused with the TRAP protein showed accumulation of TRAP in adipocytes, which show that the delivered TRAP protein is effectively taken up by the cells. This pattern was not observed in the adipocytes of the control fat pad. Measurement of adipocyte size showed a tendency to increased size distribution and infiltration of small adipocytes in treated compared to control regions, but this difference was not significant.

### Human Studies

Human abdominal subcutaneous adipose tissue was obtained from different sources. For the isolation of pre-adipocytes and mesenchymal stem cells (MSC), tissue was obtained as the product of cosmetic liposuction on otherwise healthy non-obese women (body mass index (=BMI): 22-36 kg/m²; age 22-56 years; n=10). For immunohistochemistry, tissue was obtained during surgery from three obese subjects undergoing gastric banding. For the comparison of gene and protein expression in lean versus obese subjects a frozen (−70° C.) 300 mg tissue piece was used. It was obtained by needle biopsy from lean (BMI<25 kg/m²) or obese (BMI>30 kg/m²) but otherwise healthy women participating in ongoing studies of the genetic regulation of human fat cell function. For those in the gene expression study age (mean±SD) was 36±14 and 39±7 years in lean (n=14) and obese (n=14) subjects, respectively. BMI was 23±2 and 36±4 kg/m² respectively. Fat cell volume was 450±140 and 822±240 pL in lean and obese, respectively. The obese group in the gene expression study was sub-divided in two groups according to fat cell size; hyperplasia (fat cell volume of 641±450 pL; n=7) and hypertrophia (cell volume of 1004±490 pL; n=7) with no between group difference in BMI. In the protein expression study age was 40±4 years (lean (n=7)) and 40±13 years (obese (n=12)), the BMI was 23±1 kg/m² and 38±5 kg/m², and fat cell volume was 490±190 pL and 868±223 pL, respectively. The obese group was sub-divided into a hyperplastic (fat cell volume 659±420 pL; n=6) and hypertrophic (fat cell volume 1077±570 pL; n=6) group) with no between group difference in BMI. Unpaired t-test was used to compare lean and obese subjects and adipose tissue versus adipocytes. ANOVA was used to compare lean subjects with the hypertrophic and hyperplastic groups. Degrees of freedom in these experiments were 1, 1 and 2, respectively.

### Electrophoresis and Immunoblot Analysis

Partially purified (for procedure see (Lang and Andersson, 2005)) culture media from 1×106 RAW 264.7 cells or 70 mU TRAP from WT/TRAP+p/TRAP+ adipose tissue were subjected to SDS-PAGE using 12% NuPage gels (Invitrogen, Carlsbad, Calif.) run with MOPS buffer and transferred to PVDF membranes (Bio-Rad, Hercules, Calif.).

Membranes were blocked using 1% TBST (100 mM Tris-HCl pH 7.6, 154 mM NaCl, 1% Tween-20) and stained with rabbit anti-mouse monomeric TRAP (Lang and Andersson, 2005) 1:1500 or rabbit anti rat total (Ek-Rylander et al., 1997) TRAP 1:1500 and goat anti rabbit HRP 1:10 000 (Calbiochem, La Jolla, Calif.) and developed using Renaissance (NEN Life Science, Boston, Mass.). Human samples; 100 μg of total protein total obtained from protein lysates was separated on 12% PAA-gels, transferred onto PVDF membranes and stained using rabbit anti-mouse monomeric TRAP or rabbit anti-rat total TRAP as described above. Bands were detected using Supersignal® (Pierce, Rockford, Ill.). Relative expression was determined using Chemidoc XRS System (Bio-Rad, Hercules, Calif.).

### Immunohistochemistry

Immunohistochemistry was carried out mainly as previously described (Lang and Andersson, 2005). Mouse adipose tissue from male and female mice; paraffin sections (WT; n=3, TRAP+; n=3) were treated with 0.1% trypsin (Sigma-Aldrich, St. Louis, Mo.) at 37° C. for 30 minutes and stained for macrophages using F4/80 monoclonal antibody (1:50) (Serotec, Oxford, UK). ChemMate Detection Kit Peroxidase/DAB rabbit/mouse (Dako, Glostrup, Denmark) was used as secondary antibodies and developing solution. Human adipose tissues; microwave treated (1 mM EDTA pH 8 for 10 minutes at 900 W) paraffin sections were stained with rabbit anti-rat total TRAP 1:50 (Ek-Rylander et al., 1997) recognizing both monomeric and the two-subunit TRAP or rabbit anti-mouse monomeric TRAP antibody 1:50 (Lang and Andersson, 2005) and CD68 1:100 (Dako, Glostrup, Denmark). Secondary antibodies were ALEXA 568 goat anti-rabbit Fab₂ 1:250 and ALEXA 488 goat anti-mouse Fab₂ fragments 1:100 (Invitrogen, Carlsbad, Calif.). Sections were then examined using a Leica TCS NT ArKr laser confocal microscope (Leica Microsystems AG, Wetzler, Germany).

### Expression, Purification and Cleavage of Recombinant Rat TRAP

Recombinant rat TRAP was expressed in Sf9 insect cells and purified as previously described (Wang et al., 2005). To generate the proteolytically processed form, recombinant rat TRAP was digested at 37° C. for 40 minutes with recombinant human cathepsin K in 5 mM NaOAc pH 5.5, 1 mM EDTA and 10 mM DTT using a 1:1 molar ratio.

### Proliferation and Differentiation of 3T3-L1 Cells in the Presence of TRAP

For proliferation experiments (n=4, df=3); 3T3-L1 (ATCC (LGC Promochem, Borås, Sweden)) cells (2,000 cells/cm²) were cultured in DMEM/F12 Glutamax II, 4.5 g/L glucose, 1.5 g/L sodium bicarbonate, penicillin/streptomycin, 2.5% calf bovine serum+/−cleaved (600 U/mg) or monomeric (50 U/mg) TRAP (10⁻⁹ M-10⁻¹² M). After 24, 48 and 72 h, cells were labeled for 2 h with BrdU, fixed and BrdU incorporation was measured using Cell Proliferation ELISA, BrdU kit (Roche, Mannheim, Germany).

For differentiation experiments (n=4, df=3); 3T3-L1 cells (6,000 cells/cm²) were grown into confluence in DMEM/F12 Glutamax II, 4.5 g/L glucose, 1.5 g/L sodium bicarbonate, penicillin/streptomycin, 10% calf bovine serum+/−cleaved or monomeric TRAP (10⁻⁹M-10⁻¹²M)). At 2-3 days after confluence, 0.5 mM isobutylmethylxanthine, 1 μM dexamethasone and 10 μg/ml bovine insulin was added to the media to start differentiation. Cells were then cultured+/−roziglitazone (1 μM), cleaved (600 U/mg) or monomeric (50 U/mg) TRAP (10⁻⁹M-10⁻¹²M). After 48 h, dexamethasone, isobutylmethylxanthine and roziglitazone were omitted. After an additional 48 h (i.e. 4 days after the start of differentiation), cells were lysed and GPDH activity was measured. Statistical analysis was performed using t-test.

Proliferation and Differentiation of Human Mesenchymal Stem Cells Derived from Adipose Tissue in Presence of TRAP

Human MSC were isolated from a subcutaneous lipoaspirate, grown to 60-70% confluence and passage for at least 20 passages, as described (Dicker et al., 2005). For proliferation experiments (n=3, df=2); hMSC (passage 9) (2,000 cells/cm²) were cultured in DMEM Glutamax I, 1 g/L glucose, penicillin/streptomycin, 2.5% fetal bovine serum in the presence or absence of cleaved (600 U/mg) or monomeric (50 U/mg) TRAP (10⁻⁹M-10⁻¹²M). After 48 h, cells were labeled for 2 h with BrdU, fixed and BrdU incorporation was measured as described above.

For differentiation experiments (n=3, df=2); hMSC (passage 18) were differentiated as described (Dicker et al., 2005). in the presence or absence of cleaved or monomeric TRAP (10⁻⁹ M). Twelve days after the start of differentiation cells were lysed and GPDH activity was measured. Statistical analysis was performed using t-test.

Differentiation and Proliferation of Preadipocytes Isolated from Human Adipose Tissue in Presence of TRAP.

Isolation of pre-adipocytes were performed. For proliferation experiments (n=3, df=2); cells (4000 cells/cm²) were cultured in growth medium containing 2.5% fetal bovine serum in the presence or absence of cleaved or monomeric TRAP (10⁻⁹M-10⁻¹²M). After 5 days, cells were labelled for 2 h with BrdU, fixed and BrdU incorporation was measured as described above. For differentiation experiments (n=3, df=2); cells were differentiated as described (Ryden et al., 2002). in the presence or absence of cleaved (600 U/mg) or monomeric (50 U/mg) TRAP (10⁻⁹M-10⁻¹²M). Twelve days after the start of differentiation, cells were lysed in GPDH buffer and GPDH activity was measured. Statistical analysis was performed using t-test.

Secretion of TRAP from Macrophages

RAW 264.7 cells were cultured in a 37° C. humidified 5% CO₂ atmosphere in DMEM media (Gibco, St Louis, Mo.) supplemented with 10% FCS (Gibco, St Louis, Mo.) and 0.1 mg/ml gentamycin (Gibco, St Louis, Mo.). For stimulation, 0.25×10⁶ RAW 264.7 cells/ml (passage 3) were treated with IFNγ (500 U/ml) (Invitrogen, Carlsbad, Calif.) for 16 h, and then with LPS (1 ng/μl) (Sigma-Aldrich, St. Louis, Mo.) for an additional 24 h.

### Metabolic Studies on the TRAP Over Expressing Mice

Lipolysis and lipogenesis experiments were conducted on isolated adipocytes from fat (pooled fractions of gonadal, mesenteric and inguinal fat from the same animal) from male and female mice (WT; n=8 and TRAP+; n=8). After a 2 h incubation, the cell suspension was subjected to measurement of active uptake into total fat cell lipids (lipogenesis index). Statistical analysis was performed using ANOVA.

### Measurement of Glucose, Insulin and HOMA Index

Glucose (WT; n=3 and TRAP+; n=3), HOMA index (WT; n=3 and TRAP+; n=3) and insulin (WT; n=8 and TRAP+; n=3) were determined individually on serum from male and female mice, using a Monarch automated analyzer (ILS Laboratories Scandinavia AB, Sollentuna, Sweden) or a RIA assay (Linco Research Inc., St. Charles, Mo.), respectively. Statistical analysis was performed using Mann-Whitney U test.

### Measurement of Serum Leptin, Adiponectin and TNFα

Serum levels of leptin in male and female mice >4 months of age (WT; n=26 and TRAP+; n=13) (Quantikine Mouse Leptin Immunoassay, R&D Systems, Inc., Minneapolis, Minn.), of adiponectin in male and female mice >4 months of age (WT; n=11 and TRAP+; n=7) (Adiponectin Mouse ELISA, BioVendor, Heidelberg, Germany) and of TNFα in male and female mice >4 months of age (WT; n=8 and TRAP+; n=5) (Ready-Set-Go! Mouse TNFα ELISA, ebioscience, San Diego, Calif.) was measured. Statistical analysis was carried out using Mann-Whitney U test.

### Statistical Analysis

For statistical analysis of mouse experiments with two independent group's t-test was performed. When more than two groups were present Kruskal-Wallis followed by Bonferronis correction were performed. Fat cell volume and HOMA index (log transformed to normalize values) were compared with waist using multiple regression analysis. Values are given as mean+SD. *p<0.05; **p<0.01; ***p<0.005

### Results

### Transgenic Mice Overexpressing Both Monomeric and Proteolytically Processed TRAP, But not Mainly Proteolytically Processed TRAP, Develop Spontaneous Hyperplastic Obesity

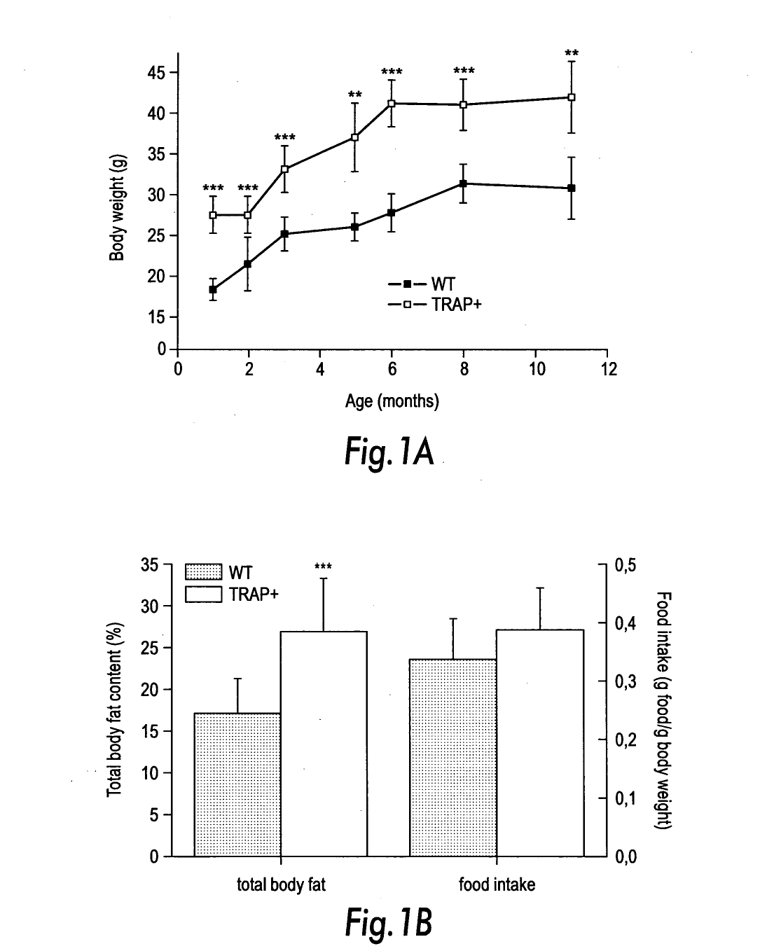

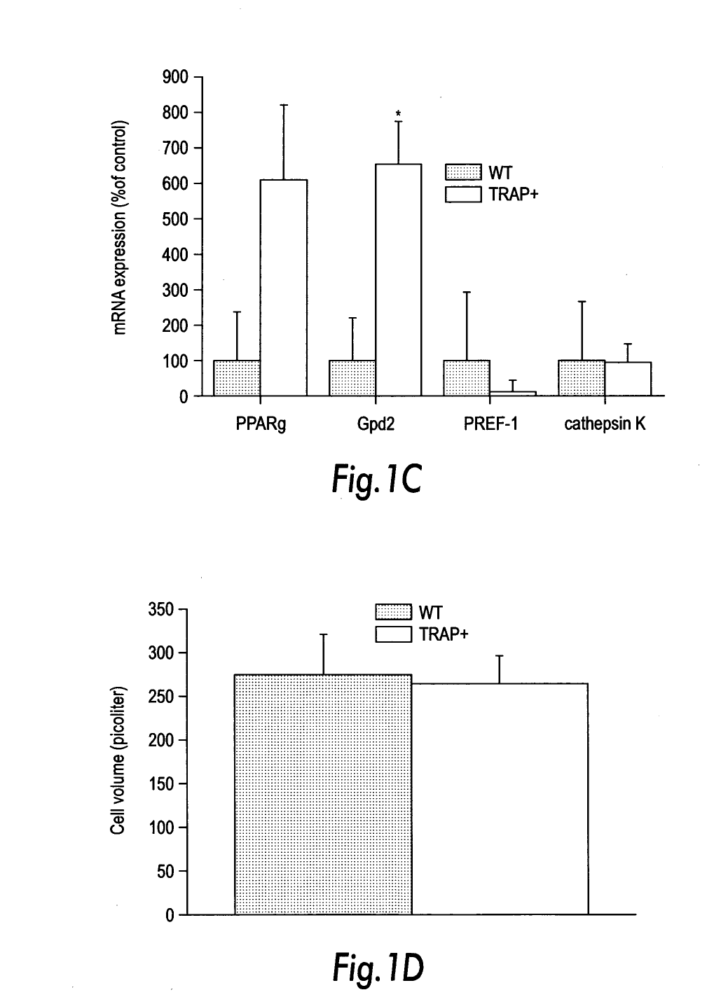

TRAP+ mice overexpressing both monomeric and proteolytically processed TRAP were found to weigh about 60% more than non-transgenic littermates (WT) already at 4 weeks of age (FIG. 1A) and this weight difference was maintained throughout the first year of life. Determination of body fat (FIG. 1B) showed a ˜60% increase of total body fat in TRAP+ mice compared to WT mice. This increase in fat content in TRAP+ mice was not associated with over-eating, since the food intake of TRAP+ and WT mice was approximately the same (FIG. 1B). Also, factors known to be associated with increased adipogenesis was either significantly upregulated (Gpd2) or showed a strong tendency towards upregulation (PPARγ) while factors known to inhibit adipogenesis (pref-1) showed a tendency towards down regulation in adipose tissue from TRAP+ mice compared to WT at the mRNA level (FIG. 1C).

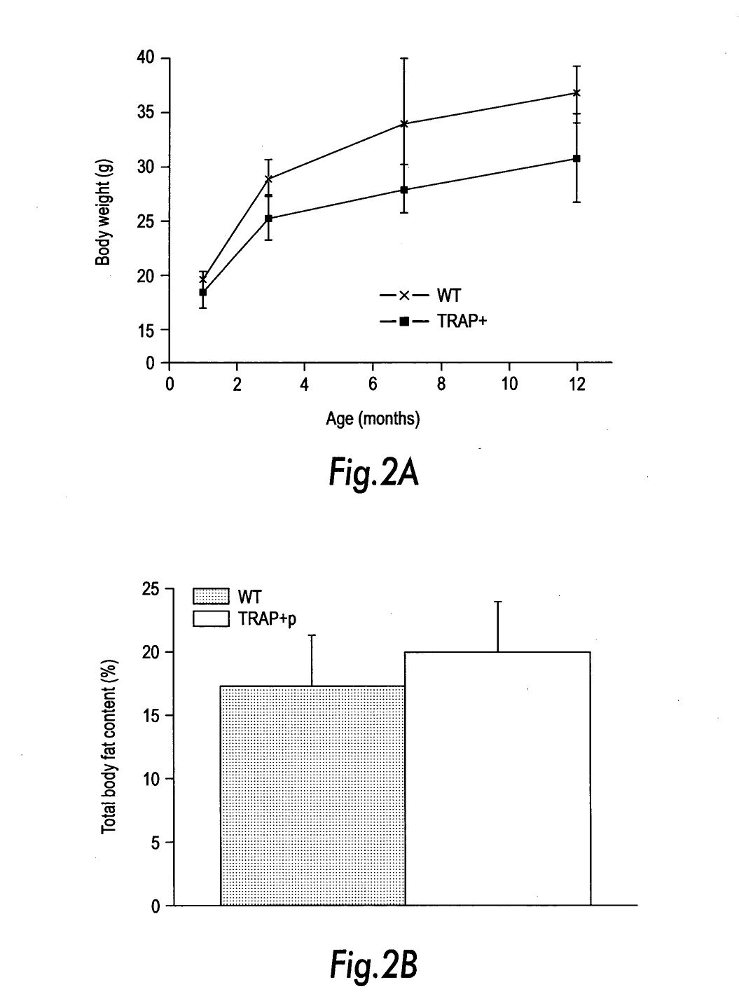

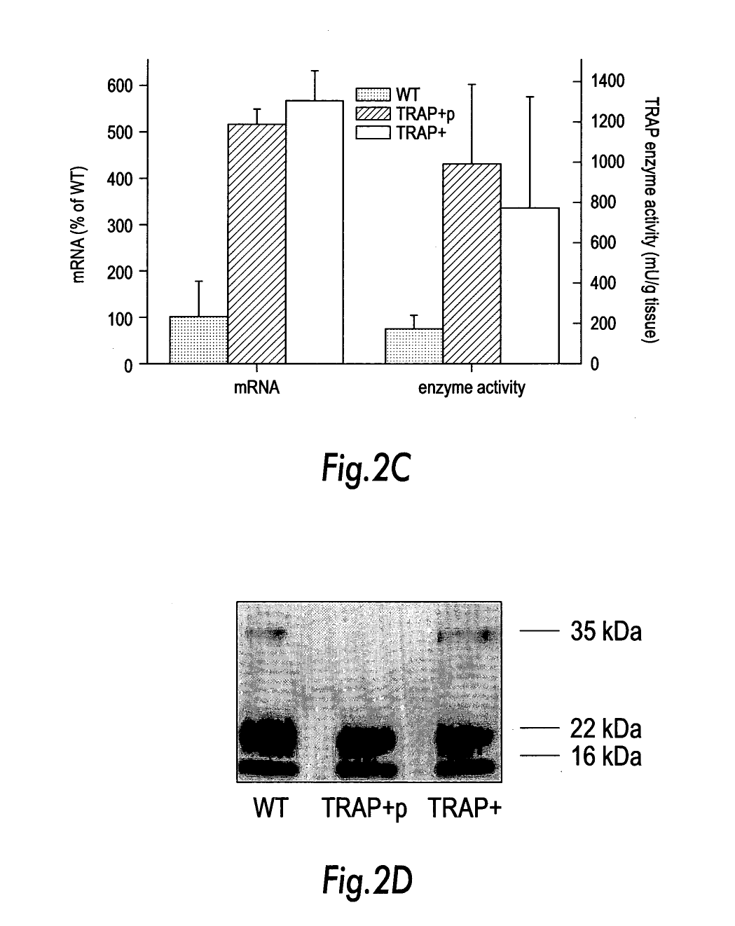

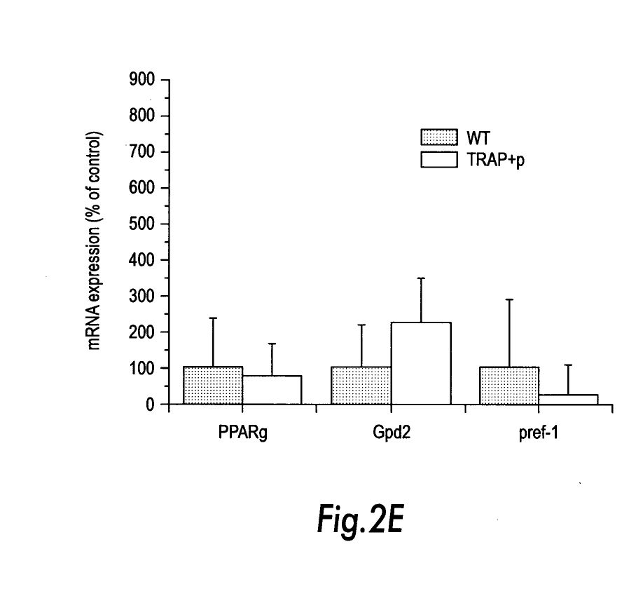

Adipose tissue can expand due to hypertrophy and/or hyperplasia of adipocytes. Hypertrophy is present in most mouse models of obesity though at least two previous models have hyperplasia as the sole cause of obesity (Shepherd et al., 1993; Valet et al., 1993). Since no difference in adipocyte cell size was found between WT and TRAP+ mice (FIG. 1D) the data indicate that overexpression of TRAP in adipose tissue promotes obesity by stimulating the formation of increased numbers of normal-sized adipocytes, i.e. induces hyperplasia. Also, the expression of cathepsin K, which has been shown to be increased in hypertrophic adipocytes (Li et al., 2002), was normal in the TRAP+ mouse. The observation that the TRAP+ animals developed marked early onset obesity without apparent over-eating indicates a strong in vivo effect of monomeric TRAP on adipogenesis. This in vivo effect of monomeric TRAP is further underscored by findings in a lean (FIG. 2A-E) transgenic substrain (TRAP+p) expressing approximately the same mRNA and enzyme activity levels of TRAP (FIG. 2C) and of the proteolytically processed form of TRAP at the protein level as the obese TRAP+ (FIG. 2D), but with clearly reduced expression of the monomeric protein (FIG. 2D) and PPARg and Gpd2 (cf FIG. 2E and FIG. 1C) in adipose tissue compared to the TRAP+ mice.

Monomeric, but not Proteolytically Processed, TRAP is Increased Both at the mRNA and Protein Level in Adipose Tissue from Obese Human Subjects

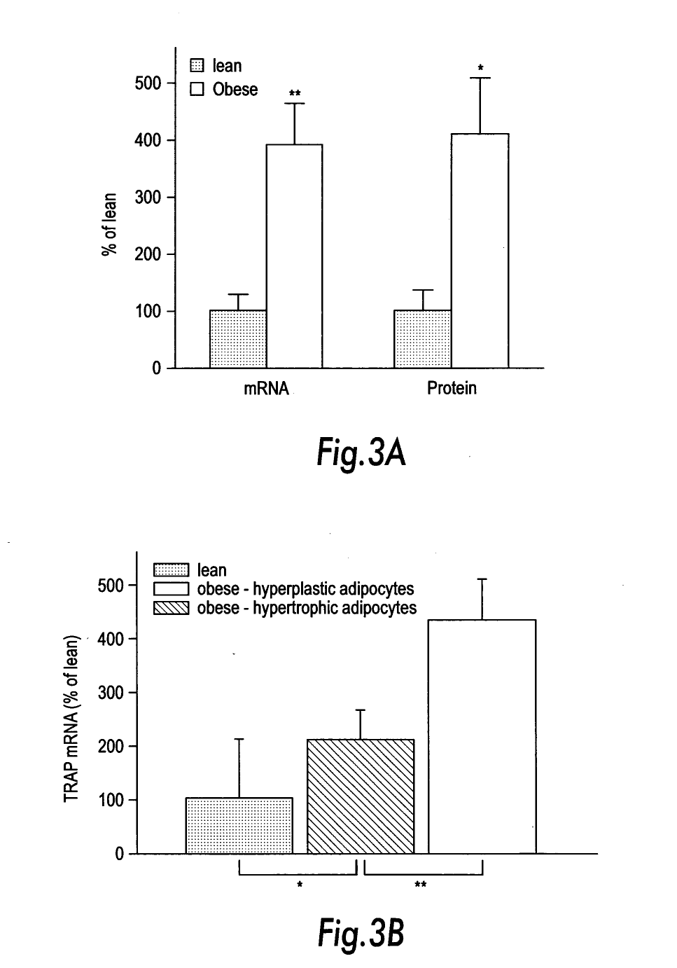

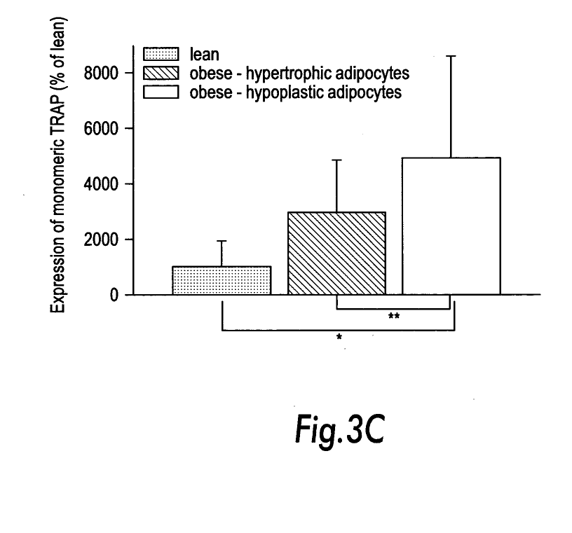

The clinical significance of TRAP as a regulator of adipogenesis was investigated in adipose tissue of lean and obese humans. Both TRAP mRNA expression and monomeric TRAP protein expression were increased by 300% among the obese subjects compared to lean subjects (FIG. 3A). In contrast, expression of proteolytically processed TRAP was similar in all three groups (data not shown) indicating a role for monomeric TRAP in adipogenesis. Stratifying the obese patient group into one group displaying a more hypertrophic phenotype and a group displaying a more hyperplastic phenotype showed that although TRAP mRNA and monomeric TRAP protein is elevated in both groups, TRAP expression is further increased in patients displaying a more hyperplastic phenotype (FIG. 3B-C).

### Monomeric, but not Proteolytically Processed, TRAP Induce Adipocyte Differentiation Ex Vivo in Pre-Adipocytes of Mouse and Human Origin

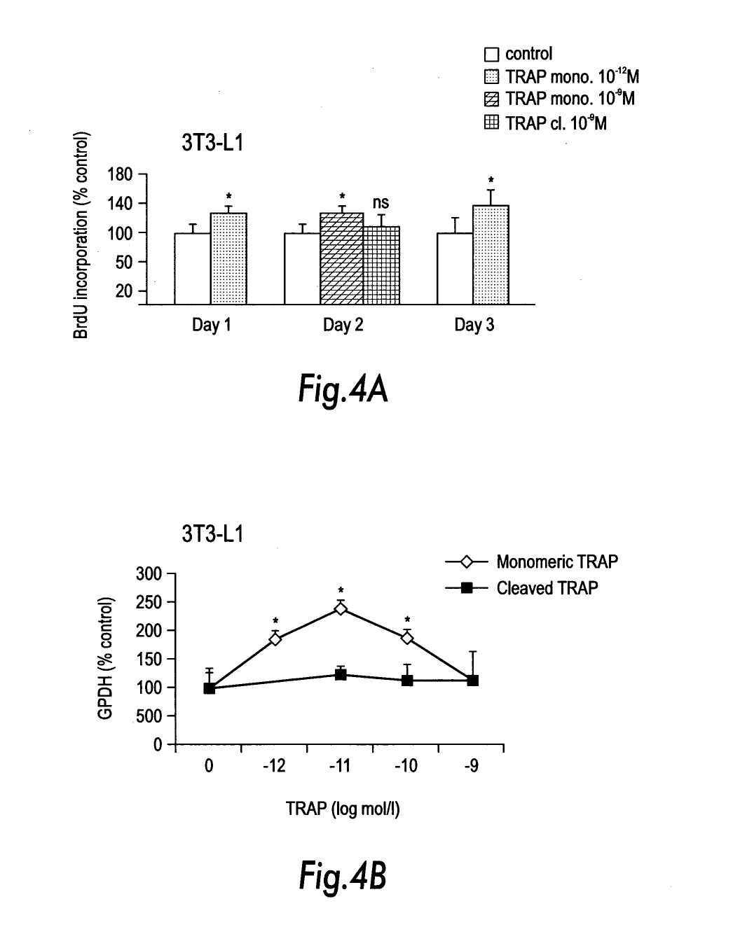

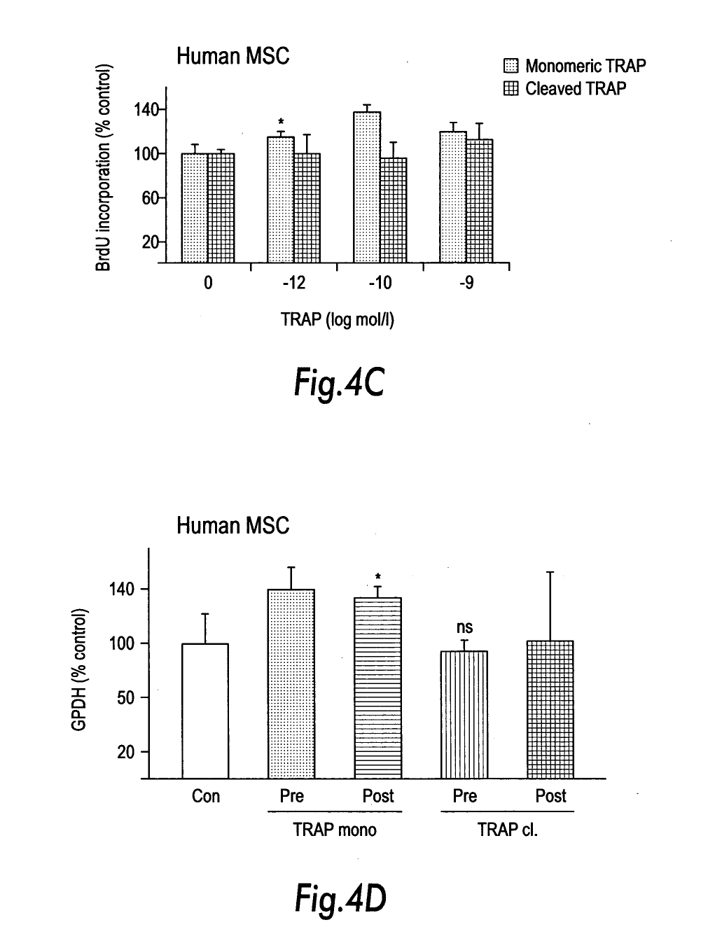

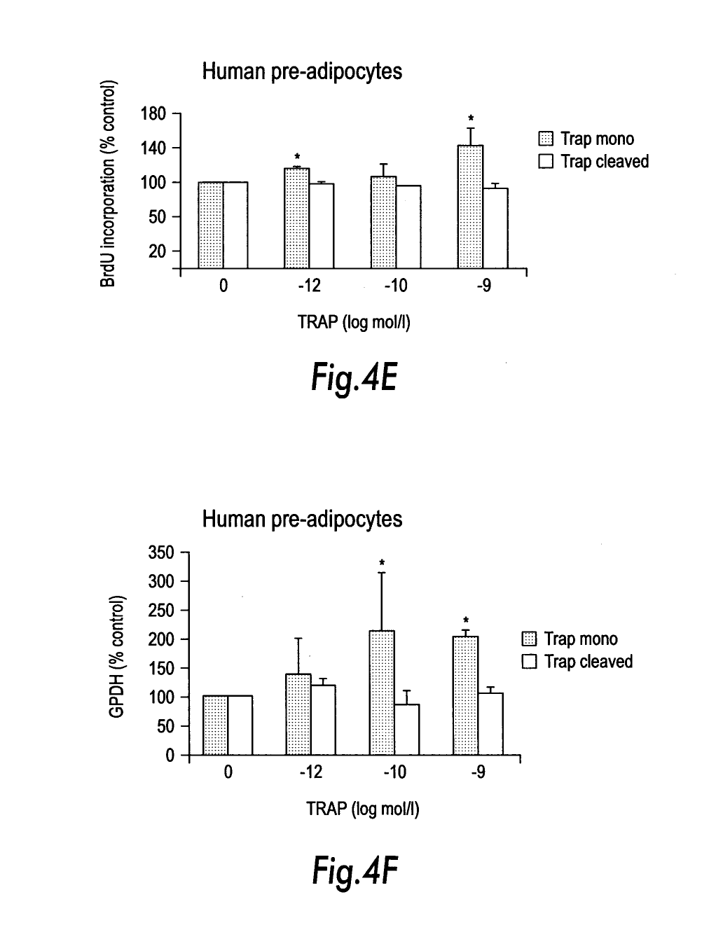

To establish if the increased adipose tissue mass in TRAP+ mice and obese human subjects could be due to, at least partly, a direct effect of TRAP on adipocytes it was demonstrated, using a mouse pre-adipocyte cell line (3T3-L1), that monomeric TRAP, at 10-11-10-12 M, caused a 30% enhancement of cell proliferation (FIG. 4A) and a 250% increase of terminal adipocyte differentiation (FIG. 4B). In contrast, proteolytically processed TRAP affected neither proliferation nor differentiation. Similar differential effects of monomeric vs. proteolytically processed TRAP were also observed in adipocytes derived from human MSCs (FIG. 4C-D) as well as human pre-adipocytes (FIG. 4E-F), although at somewhat higher concentrations. The differentiation effect is indistinguishable in the presence (FIG. 4A-D) or absence (data not shown) of the PPAR-gamma activator, roziglitazone (a glitazone) suggesting that monomeric TRAP is adipogenic at all levels of PPAR-gamma activity.

### Macrophages Secrete and are the Major Source of Monomeric TRAP in Mouse and Human Adipose Tissue.

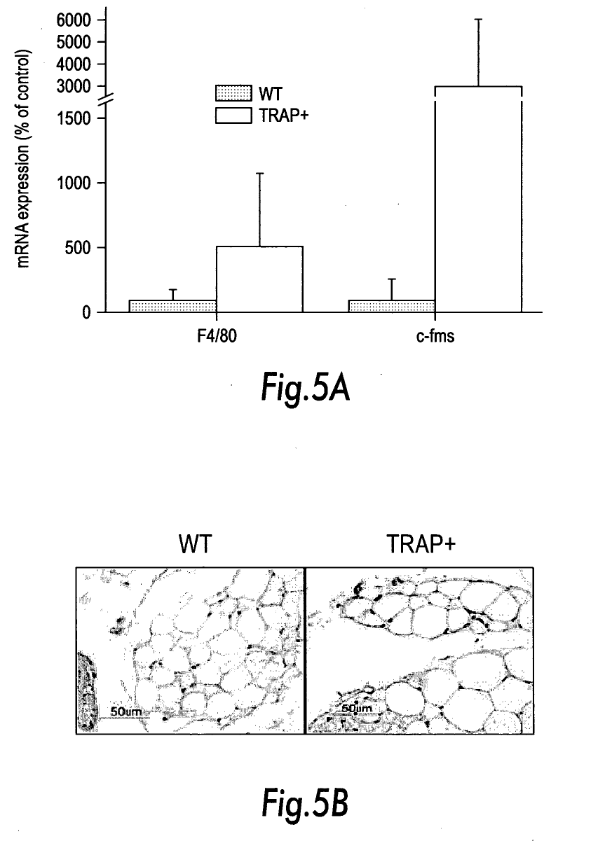

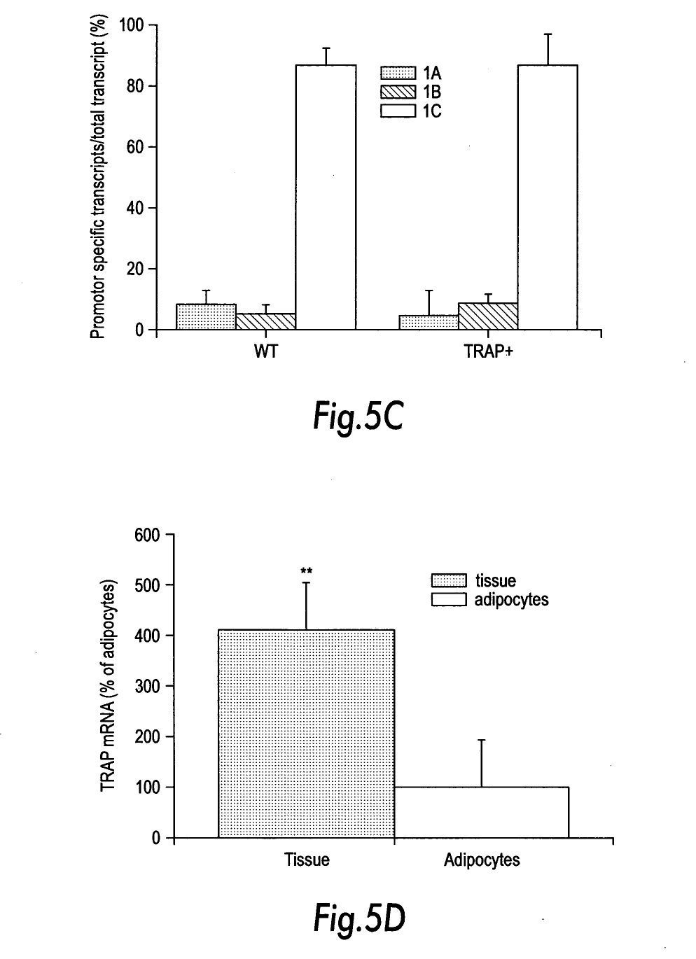

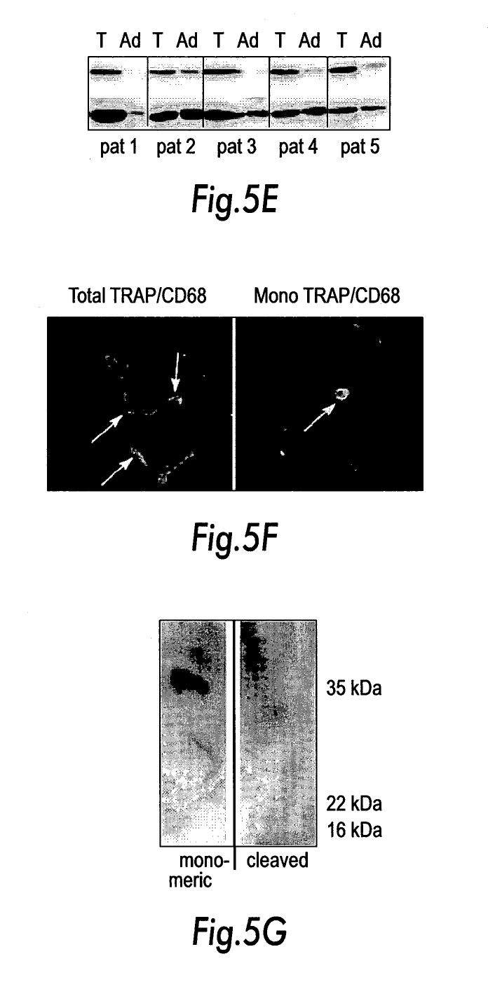

Next, an effort was made to identify the cell type responsible for the production of monomeric TRAP in adipose tissue. In line with previous studies in animal models of obesity (Weisberg et al., 2003; Xu et al., 2003) mRNA for F4/80 and c-fms (FIG. 5A) as well as F4/80 staining (FIG. 5B) were increased in gonadal adipose tissue from TRAP+ mice indicative of an increase of infiltrating macrophages. Since TRAP is present mainly in myeloid lineage cells, it was investigated whether the increase of TRAP mRNA and enzyme activity in TRAP+ adipose tissue (FIG. 1A-B) could be due to the increased number of macrophages. 87% of TRAP mRNA transcripts in both TRAP+ and WT mice are derived from the myeloid lineage-specific TRAP transcript 1C (Walsh et al., 2003) (FIG. 5C). In human adipose tissue TRAP was mainly expressed by cells in the stroma cell fraction (FIG. 5D-E) identified by immunohistochemistry as CD68 positive macrophages (FIG. 5F) supporting the idea that macrophages are the primary source of TRAP in mouse and human adipose tissue. If macrophages are the primary source, TRAP must be secreted from these cells in order to affect adipocytes. Confirming previous findings (Janckila et al., 2005) it is shown that the mouse macrophage cell line RAW 264.7 stimulated with LPS and IFN-γ is able to secrete monomeric TRAP (FIG. 5G). This suggests that obesity is associated with increased numbers of macrophages in adipose tissue, which by secreting monomeric TRAP stimulate proliferation and differentiation of adipocytes.

### Transgenic Mice Overexpressing TRAP Exhibit a Partly Altered Expression Profile of Adipokines and Cytokine in Adipose Tissue and Serum

Since increased total body weight due to increased total body fat i.e. obesity, is known to be associated with a low-grade inflammation serum protein levels and/or mRNA levels for inflammatory markers known to be increased in obesity were measured in TRAP+ mice. The present inventors investigated three groups of factors, those mainly expressed by adipocytes (adiponectin, leptin, CCL2) (Bouloumie et al., 2005; Dahlman et al., 2005), those believed to be mainly expressed by cells from the stroma cell fraction i.e. likely macrophages (TNFα, IL1β, MMP9) (Bouloumie et al., 2005) and those believed to be expressed in both adipocytes and the stroma cell fraction (IL6) (Bouloumie et al., 2005). The mRNA levels of leptin and adiponectin was found to be unchanged between the TRAP+ and its WT littermates although the ratio between adiponectin and leptin is slightly shifted in favour of leptin. However, the serum leptin levels where increased in the TRAP+ mouse compared to WT mice leading to a non-significant shift in the ratio between adiponectin and leptin in favour of leptin. Also the mRNA levels of TNFα was increased in TRAP+ mice compared to WT littermates although serum TNFα only showed a non-significant increase (data not shown). On the other hand, neither CCL2, MMP9, IL1β nor IL6 was affected in the TRAP+ mice.

Adipocytes from Transgenic Mice Overexpressing TRAP are Metabolically Normal and the Animals has No Signs of Decreased Insulin Sensitivity

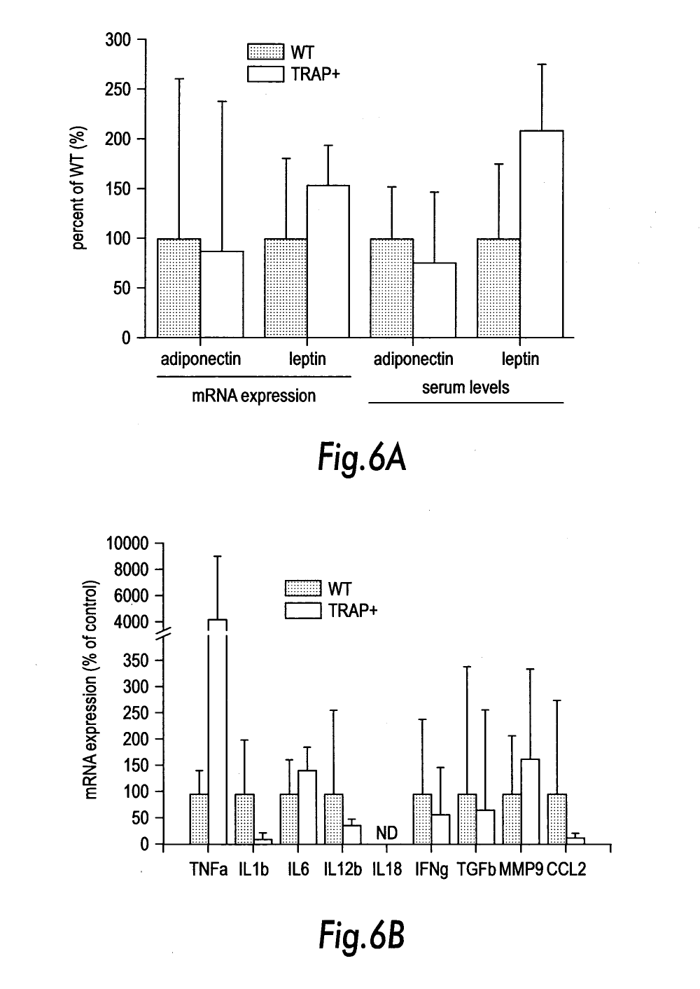

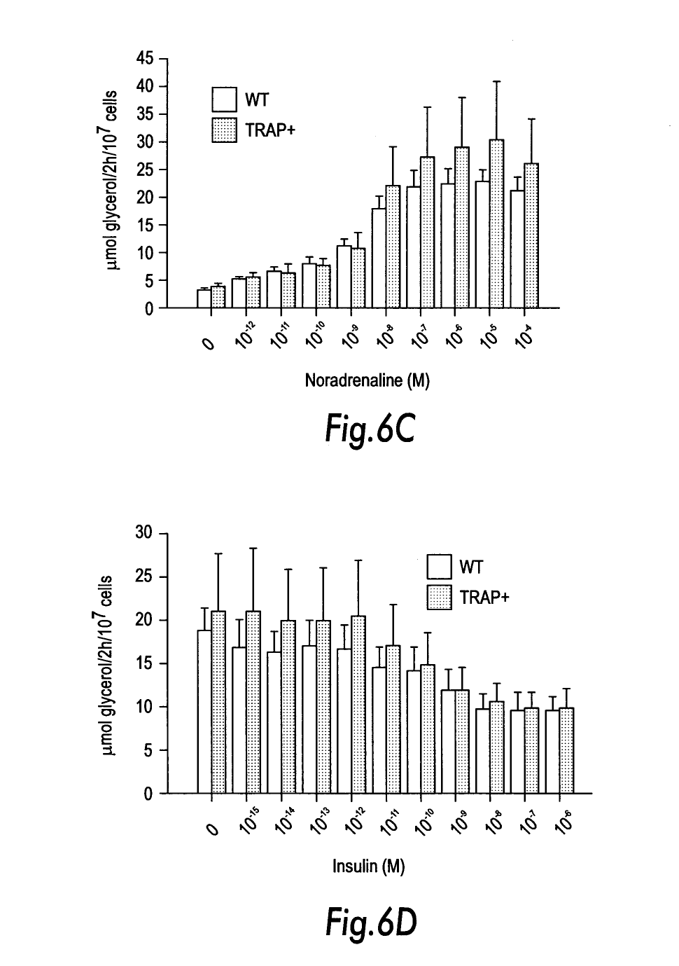

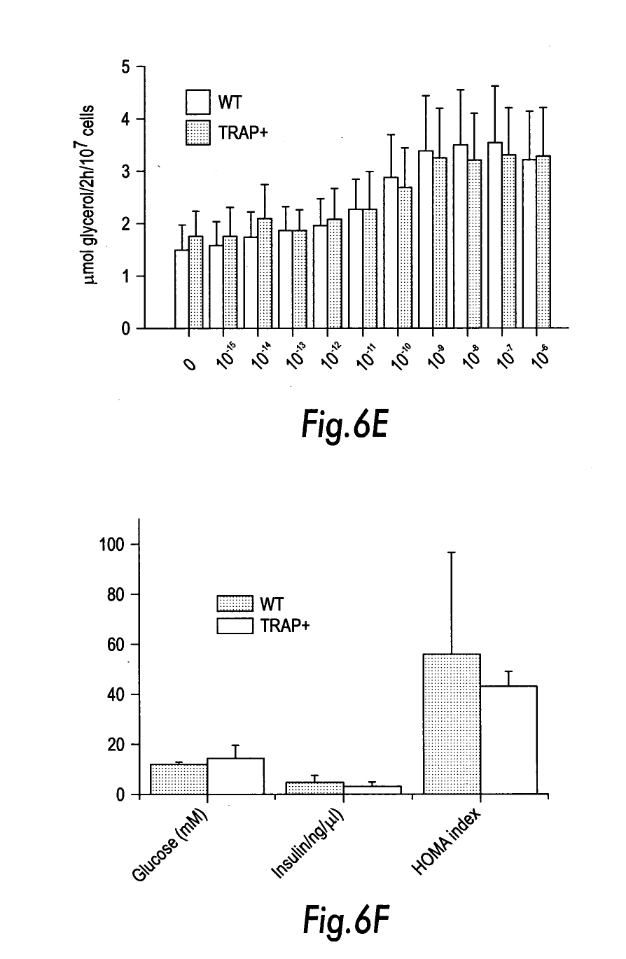

It is well known that insulin and catecholamine actions are altered in obesity which, at least in part, can be linked to the increased fat cell size seen in most models of obesity. However, adipocytes isolated from TRAP+ mice exhibited normal spontaneous basal lipolysis (FIG. 6C) as well as lipogenesis (FIG. 6D) and the effect of maximum effective concentrations of noradrenalin (on lipolysis) (FIG. 6E) and insulin (on lipogenesis and lypolysis) (FIG. 6F) was also not affected in the transgenic mice. Half maximum effective concentrations of the hormone were also similar in adipocytes from WT and TRAP+ mice (data not shown).

### Obese Human Subjects Displaying a More Hyperplastic Obesity is Less Insulin Resistant Than Human Subjects Displaying a More Hypertrophic Obesity

To explore the importance of fat cell size for in vivo insulin sensitivity the present inventors analysed the relationship between abdominal subcutaneous fat cell volume and HOMA-index, the latter a well established indirect measurement of insulin sensitivity in vivo. For this purpose, unpublished data were obtained from a previous study on 112 healthy women with a large interindividual variation in BMI (Wahrenberg et al., 2005). It had been previously demonstrated that waist is the strongest predictor for HOMA index (Wahrenberg et al., 2005). In the whole material and in the obese subgroup fat cell volume was an independent predictor for HOMA index, taking in account the influence of waist (cf. Table 2 herein below). In non obese only waist was a significant predictor. Thus, in obese subjects, the fat cell size is an independent determinator of insulin independent of the degree of fatness (measured as waist). Obese subjects with hyperplasia seem more insulin sensitive than obese subjects with adipocyte hypertrophy.

TABLE 2
Multiple regression for waist and fat cell volume
versus log HOMA index in healthy women.

| Group | Measure | Partial r | P-value |
|---|---|---|---|
| Non-obese (n = 146) | Fat cell volume | 0.056 | 0.59 |
| | Waist | 0.41 | <0.0001 |
| Obese (n = 227) | Fat cell volume | 0.201 | 0.0016 |
| | Waist | 0.4 | <0.0001 |
| All (n = 373) | Fat cell volume | 0.213 | 0.0002 |
| | Waist | 0.54 | <0.0001 |

### Discussion

### Mice Overexpressing TRAP Develop Hyperplastic Obesity

Mice engineered to overexpress TRAP presented an early onset of an obese phenotype with significantly increased body fat content that was not due to apparent over-eating, indicating that TRAP may have a direct effect on adipocytes. Adipose tissue can expand either by lipid filling of existing adipocytes resulting in large hypertrophic adipocytes or by increased differentiation of normally sized adipocytes resulting in hyperplastic obesity. In the TRAP overexpressing mouse adipocyte volume was normal and genes known to increase adipocyte differentiation (PPARγ, Gpd2) were either upregulated or showed such a tendency, whereas genes inhibiting adipocyte differentiation (pref-1) showed a tendency towards downregulation suggesting that the increased fat mass was largely due to enhanced differentiation and/or proliferation of adipocytes rather than lipid filling of pre-existing adipocytes, i.e. the TRAP+mouse develops hyperplastic obesity. This makes the model atypical since hyperthrophy is dominating in most mouse models of obesity though at least two previous models have hyperplasia as the major cause of obesity (Shepherd et al., 1993; Valet et al., 1993). The results from the TRAP overexpressing mouse also suggests that the monomeric form of TRAP is causing the effect of TRAP on adipogenesis, since a sub-strain of the mouse only expressing proteolytically processed TRAP in adipose tissue was lean. The mechanism underlying this difference between the sub-strains is unclear although it clearly illustrates the importance of the monomeric rather than the proteolytically processed form of TRAP for the development of the obese phenotype.

Monomeric TRAP is Increased in Patients Suffering from Obesity

The finding that TRAP overexpressing mice develops spontaneous hyperplastic obesity lead the present inventors to investigate if increase of TRAP is associated also with human obesity and, in fact, TRAP mRNA was increased in patients suffering from obesity. In consistence with the findings in the TRAP overexpressing mouse an increased expression of monomeric TRAP was found, but not proteolytically processed TRAP, in adipose tissue of obese subjects. Human obesity is usually characterized by a combination of hypertrophic adipocytes and an increased number of hyperplastic adipocytes, although hypertrophy usually dominates (Bonnet et al., 1979; Brook et al., 1972). However, some patients do present an obese phenotype with a higher percentage of hyperplastic (i.e. smaller than expected) adipocytes (Hirsch et al., 1989). When obese subjects were divided into a group with a shift towards hyperplastic obesity and one with a more pronounced hypertrophic obesity both TRAP mRNA and monomeric TRAP were increased in subjects with hypertrophic obesity compared to lean subjects but the expression was further increased in those with hyperplastic obesity. This indicates that monomeric TRAP is associated with enhanced formation of adipocytes, and thereby hyperplasia, also in human obesity.

### Monomeric TRAP Induces Proliferation and Differentiation of Mice and Human Pre- and Adipocytes In Vitro

The direct influence of TRAP on development of obesity in the TRAP overexpressing mice and in human obesity was subsequently addressed by experiments on mouse and human adipocyte progenitors where the potency of monomeric TRAP to enhance adipocyte proliferation as well as differentiation was assessed. In both hMSC and pre-adipocytes of mouse and human origin, monomeric TRAP stimulated proliferation weakly but exerted a stronger potentiation on differentiation. The effect on differentiation was indistinguishable in the presence or absence of the PPARy activator roziglitazone suggesting that the mechanisms for PPARy and monomeric TRAP signalling are not directly coupled. However, proteolytically processed TRAP had no effect on either proliferation or differentiation of adipocytes. This dual effect on both proliferation and differentiation increases the impact of monomeric TRAP although the effect at each stage may appear small, at least in respect to stimulation of proliferation. These combined results from the TRAP overexpressing mouse, obese human subjects and ex vivo proliferation and differentiation of pre-adipocytes suggest a potentially important functional action on adipogenesis of monomeric TRAP which hitherto has been recognized only as a latent pro-enzyme (Ljusberg et al., 1999).

### Macrophages Secrete and are the Primary Source of TRAP in Adipose Tissue

Obesity has been found to be associated with a low-grade inflammation in adipose tissue (Fantuzzi, 2005; Wellen and Hotamisligil, 2003). This inflammation is characterized by an influx of macrophages into the adipose tissue (Weisberg et al., 2003; Xu et al., 2003). The increased influx of macrophages has been hypothesized to be a consequence of adipocyte necrosis because of adipocyte hypertrophy (Cinti et al., 2005) and/or increase of monocyte attractants such as MCP-1/CCL2 (Dahlman et al., 2005; Kamei et al., 2006; Kanda et al., 2006). When in the adipose tissue, these macrophages secrete different cytokines that affects the adipocytes for example TNFα (Uysal et al., 1997). Given that TRAP is normally expressed in cells from the myeloid linage (Hayman et al., 2000; Hayman et al., 2001; Lang and Andersson, 2005) the present inventors investigated if TRAP was expressed in this population also in adipose tissue of mouse and human origin, thus indicating a paracrine effect of monomeric TRAP on adipocytes. In mouse, almost 90% of the TRAP transcripts originate from the myeloid specific transcript 1C and in human tissue, TRAP was mainly expressed in CD68 positive macrophages. In vitro studies also confirmed previously published data (Janckila et al., 2005) that monomeric TRAP can be secreted from macrophage cell lines in vitro. In light of these data, a hypothesis is proposed in which macrophages controls the development of obesity by directly inducing adipogenesis. It is hypothesized that in a situation where there is an increased calorie intake resulting in increased adipose tissue mass and thereby macrophage influx, macrophages secreting monomeric TRAP induces adipogenesis by an, at least partly, PPARγ independent pathway, thus contributing to the increase of total adipose tissue mass.

Consequently, if this increase in secretion of monomeric TRAP is persistent, novel and small fat cells are formed contributing to the hyperplasia component of obese adipose tissue.

Consequences of Increased Expression and Secretion of TRAP from Macrophages in Adipose Tissue−Normal Adipocyte Size=Normal Metabolism, Gene Expression Profile and Insulin Sensitivity

Obesity is linked to insulin resistance (Hubert et al., 1983; Kissebah et al., 1982) and several studies have highlighted the correlation between adipocyte size rather than adipose tissue mass and insulin malfunction (Brook and Lloyd, 1973; Kissebah et al., 1982; Salans et al., 1973; Salans et al., 1968; Stern et al., 1972; Weyer et al., 2000). To explain this correlation at least two theories have been presented. One is that enlargement of adipocytes does not have pathophysiological significance by itself but is rather a manifestation of other pathogenic factors leading to insulin resistance (Jernas et al., 2006; Weyer et al., 2000; Winkler et al., 2003). One such factor is increased adipocyte lipolysin resulting in elevated fatty acids, which in turn cause insulin resistance (Amer, 2003). The second theory implies that enlarged adipocytes are themselves pathogenic for example by a change of gene expression profile involving, among others, altered expression of adipokines and cytokines (Weyer et al., 2000). Since the obese TRAP+ mice had normal fat cell size, their adipocyte metabolism, degree of insulin resistance and alteration in gene expression profile in adipose tissue were investigated. As judged by circulating insulin and glucose levels and by measurement of insulin action of lipolysis and lipogenesis, isolated fat cells from the obese TRAP+ mouse exhibited normal insulin sensitivity, although it can not be excluded that some alterations in insulin action in skeletal muscle or liver which were not directly examined. Furthermore, basal and catecholamine induced lipolysis were also normal in the obese mice. Thus, there was no evidence of enhanced expression of fatty acid mobilisation genes in adipose tissue in the TRAP+mouse. An increased amount of TNFα mRNA (though serum TNFα levels appeared normal) but no change in other cytokines such as IL1b and IL6 or the ratio between leptin and adiponectin were found. Thus, the absence of fat cell hypertrophy and the modest change in expression of cytokines associated with an innate immune response most likely explain why obese TRAP+mice had nor or little evidence of insulin resistance. The relation of fat cell size for in vivo insulin sensitivity in human also was investigated since previously published data suggests a relationship between these factors (Brook and Lloyd, 1973; Kissebah et al., 1982; Salans et al., 1973; Salans et al., 1968; Stern et al., 1972; Weyer et al., 2000). It was found that fat cell size is related to in vivo insulin sensitivity and this is independent of degree of adiposity only among the obese. Thus, it appears that in both man and mice the hyperplastic component of obesity is protective against insulin resistance. In summary, it appears that macrophage infiltration of adipose tissue is important for the formation of new and small fat cell during development of obesity. The mechanism by which macrophages increase proliferation and differentiation of adipocyte precursor cells is presumably by secretion of monomeric TRAP. These newly formed fat cells have normal lipid metabolism that makes the tissue prone to accumulate lipid.

### REFERENCES

### Non Patent Documents

American Psychiatric Association Work Group on Eating Disorders (2000). Practice guideline for the treatment of patients with eating disorders (revision). American Journal of Psychiatry, 157(1 Suppl): 1-39.
Andersson, G., Ek-Rylander, B., Hollberg, K., Ljusberg-Sjolander, J., Lang, P., Norgard, M., Wang, Y., and Zhang, S. J. (2003). TRACP as an osteopontin phosphatase. J Bone Miner Res 18, 1912-1915.
Angel, N. Z., Walsh, N., Forwood, M. R., Ostrowski, M. C., Cassady, A. I., and Hume, D. A. (2000). Transgenic mice overexpressing tartrate-resistant acid phosphatase exhibit an increased rate of bone turnover. J Bone Miner Res 15, 103-110.
Arner, P. (2003). The adipocyte in insulin resistance: key molecules and the impact of the thiazolidinediones. Trends Endocrinol Metab 14, 137-145.
Arner, P., Wahrenberg H., Lonnqvist F., Angelin B. (1993). Adipocyte beta-adrenoceptor sensitivity influences plasma lipid levels. Arterioscler. Thromb. Vasc. Biol.
Barber M D, Ross J A, Voss A C, Tisdale M J, Fearon K C. (1999). The effect of an oral nutritional supplement enriched with fish oil on weight loss in patients with pancreatic cancer. Br J Cancer; 81:80-6.
Bonnet, F. P., Rocour-Brumioul, D., and Heuskin, A. (1979). Regional variations of adipose cell size and local cellularity in human subcutaneous fat during normal growth. Acta Paediatr Belg 32, 17-27.
Bouloumie, A., Curat, C. A., Sengenes, C., Lolmede, K., Miranville, A., and Busse, R. (2005). Role of macrophage tissue infiltration in metabolic diseases. Curr Opin Clin Nutr Metab Care 8, 347-354.
Brook, C. G., and Lloyd, J. K. (1973). Adipose cell size and glucose tolerance in obese children and effects of diet. Arch Dis Child 48, 301-304.
Brook, C. G., Lloyd, J. K., and Wolf, O. H. (1972). Relation between age of onset of obesity and size and number of adipose cells. Br Med J 2, 25-27.
Bruera, E., Roca, E., Cedaro, L., Carraro, S., Chacon, R. (1985) Action of oral methylprednisolone in terminal cancer patients: a prospective randomized double-blind study. Cancer Treat Rep 1985; 69:751-4.
Cinti, S., Mitchell, G., Barbatelli, G., Murano, I., Ceresi, E., Faloia, E., Wang, S., Fortier, M., Greenberg, A. S., and Obin, M. S. (2005). Adipocyte death defines macrophage localization and function in adipose tissue of obese mice and humans. J Lipid Res 46, 2347-2355.
Dagnelie P C, Bell J D, Williams S C, Bates T E, Abel P D, Foster C S. (1994). Effect of fish oil on cancer cachexia and host liver metabolism in rats with prostate tumors. Lipids; 29:195-203.
Dahlman, I., Kaaman, M., Olsson, T., Tan, G. D., Bickerton, A. S., Wahlen, K., Andersson, J., Nordstrom, E. A., Blomqvist, L., Sjogren, A., et al. (2005). A unique role of monocyte chemoattractant protein 1 among chemokines in adipose tissue of obese subjects. J Clin Endocrinol Metab 90, 5834-5840.
Della Cuna, G R., Pellegrini, A., Piazzi, M. (1989). Effect of methylprednisolone sodium succinate on quality of life in preterminal cancer patients: a placebo-controlled multicenter study. Eur J Cancer Clin Oncol; 25:1817-21
Dicker, A., Le Blanc, K., Astrom, G., van Harmelen, V., Gotherstrom, C., Blomqvist, L., Arner, P., and Ryden, M. (2005). Functional studies of mesenchymal stem cells derived from adult human adipose tissue. Exp Cell Res 308, 283-290.
Downer S, Joel S, Allbright A, Plant H, Stubbs L, Talbot D, Slevin M. (1993) A double blind placebo controlled trial of medroxyprogesterone acetate (MPA) in cancer cachexia. Br J Cancer; 67:1102.
Durnin J. V. G. A. and Rahaman, M. M. (1967). The assessment of the amount of fat in the human body from the measurement of Skinfold Thickness. Br. J. Nutr 21, 681-688.
Ek-Rylander, B., Barkhem, T., Ljusberg, J., Öhman, L., Andersson, K. K., and Andersson, G. (1997). Comparative studies of rat recombinant purple acid phosphatase and bone tartrate-resistant acid phosphatase. Biochem J 321, 305-311.
Fantuzzi, G. (2005). Adipose tissue, adipokines, and inflammation. J Allergy Clin Immunol 115, 911-919; quiz 920.
Fonesca, V. (2003). Effect of thiazolidinediones on body weight in patients with diabetes mellitus. Am J Med 115: 42S-48S.
Halleen, J., Raisanen, S., Salo, J., Reddy, S., Roodman, G., Hentunen, T., Lehenkari, P., Kaija, H., Vihko, P., and Vaananen, H. (1999). Intracellular fragmentation of bone resorption products by reactive oxygen species generated by osteoclastic tartrate-resistant acid phosphatase. J Biol Chem 274, 22907-22910.
Hayman, A., Bune, A., Bradley, J., Rashbass, J., and Cox, T. (2000). Osteoclastic tartrate-resistant acid phophatase (Acp 5): its to dendritic cells and diverse murine tissues. J Histochem Cytochem 48, 219-228.
Hayman, A. R., Macary, P., Lehner, P. J., and Cox, T. M. (2001). Tartrate-resistant acid phosphatase (Acp 5): identification in diverse human tissues and dendritic cells. J Histochem Cytochem 49, 675-684.
Hirsch, J., Fried, S. K., Edens, N. K., and Leibel, R. L. (1989). The fat cell. Med Clin North Am 73, 83-96.
Hoffstedt, J., Arvidsson, E., Sjolin, E., Wahlen, K., and Amer, P. (2004). Adipose tissue adiponectin production and adiponectin serum concentration in human obesity and insulin resistance. J Clin Endocrinol Metab 89, 1391-1396.
Hollberg, K., Nordahl, J., Hultenby, K., Mengarelli-Widholm, S., Andersson, G., and Reinholt, F. P. (2005). Polarization and secretion of cathepsin K precede tartrate-resistant acid phosphatase secretion to the ruffled border area during the activation of matrix-resorbing clasts. J Bone Miner Metab 23, 441-449.
Hubert, H. B., Feinleib, M., McNamara, P. M., and Castelli, W. P. (1983). Obesity as an independent risk factor for cardiovascular disease: a 26-year follow-up of participants in the Framingham Heart Study. Circulation 67, 968-977.
Janckila, A. J., Parthasarathy, R. N., Parthasarathy, L. K., Seelan, R. S., Hsuch, Y. C., Rissanen, J., Alatalo, S. L., Halleen, J. M., and Yam, L. T. (2005). Properties and expression of human tartrate-resistant acid phosphatase isoform 5a by monocyte-derived cells. J Leukoc Biol 77, 209-218.
Jernas, M., Palming, J., Sjoholm, K., Jennische, E., Svensson, P. A., Gabrielsson, B. G., Levin, M., Sjogren, A., Rudemo, M., Lystig, T. C., et al. (2006). Separation of human adipocytes by size: hypertrophic fat cells display distinct gene expression. Faseb J 20, 1540-1542.
Kamei, N., To be, K., Suzuki, R., Ohsugi, M., Watanabe, T., Kubota, N., Ohtsuka-Kowatari, N., Kumagai, K., Sakamoto, K., Kobayashi, M., et al. (2006). Overexpression of MCP-1 in adipose tissues causes macrophage recruitment and insulin resistance. J Biol Chem.
Kanda, H., Tateya, S., Tamori, Y., Kotani, K., Hiasa, K., Kitazawa, R., Kitazawa, S., Miyachi, H., Maeda, S., Egashira, K., and Kasuga, M. (2006). MCP-1 contributes to macrophage infiltration into adipose tissue, insulin resistance, and hepatic steatosis in obesity. Clin Invest 116, 1494-1505.
Kardinal C G, Loprinzi C L, Schaid D J, Hass A C, Dose A M, Athmann L M, Mailliard J A, McCormack G W, Gerstner J B, Schray M F. (1990). A controlled trial of cyproheptadine in cancer patients with anorexia and/or cachexia. Cancer; 65:2657-62.
Kissebah, A. H., Vydelingum, N., Murray, R., Evans, D. J., Hartz, A. J., Kalkhoff, R. K., and Adams, P. W. (1982). Relation of body fat distribution to metabolic complications of obesity. J Clin Endocrinol Metab 54, 254-260.
Lang, P., and Andersson, G. (2005). Differential expression of monomeric and proteolytically processed forms of tartrate-resistant acid phosphatase in rat tissues. Cell Mol Life Sci 62, 905-918.
Lang, P., Schultzberg, M., and Andersson, G. (2001). Expression and distribution of tartrate-resistant purple acid phosphatase in the rat nervous system. J Histochem Cytochem 49, 379-396.
Li, J., Yu, X., Pan, W., and Unger, R. H. (2002). Gene expression profile of rat adipose tissue at the onset of high-fat-diet obesity. Am J Physiol Endocrinol Metab 282, E1334-1341.
Liedman, B., Andersson, H., Bosaeus, I. Hugosson, I., Lundell, L., (1997). Changes in Body Composition after Gastrectomy: Results of a Controlled, Prospective Clinical Trial. World J Surg 21, 416-421.
Ljusberg, J., Ek-Rylander, B., and Andersson, G. (1999). Tartrate-resistant purple acid phosphatase is synthesized as a latent proenzyme and activated by cysteine proteinases. Biochem J 343 Pt 1, 63-69.
Ljusberg, J., Wang, Y., Lang, P., Norgard, M., Dodds, R., Hultenby, K., Ek-Rylander, B., and Andersson, G. (2005). Proteolytic Excision of a Repressive Loop Domain in Tartrate-resistant Acid Phosphatase by Cathepsin K in Osteoclasts. J Biol Chem 280, 28370-28381.
Lundholm, K., Gelin, J., Hyltander, A., Lönnroth, C., Sandström, R., Svaninger, G., with support from Körner, U., Gülich M., Kärrefors, I., Norli, B., Hafström, L. O., Kewenter, J., Olbe, L., Lundell, L. (1994). Anti-inflammatory treatment may prolong survival in undernourished patients with metastatic solid tumors. Cancer Res; 54:5602-6.
Maltoni, M., Nanni, O., Scarpi, E., Rossi, D., Serra, P, Amadori, D. (2001) High-dose progestins for the treatment of cancer anorexia-cachexia syndrome: a systematic review of randomized clinical trials. Ann Oncol; 12:289-300.
McMillan, D C, Wigmore, S J, Fearon K C, O'Gorman P, Wright C E, McArdle C S. (1999) A prospective randomized study of megestrol acetate and ibuprofen in gastrointestinal cancer patients with weight loss. Br J Cancer; 79:495-500.
Nagy, T. R., and Clair, A. L. (2000). Precision and accuracy of dual-energy X-ray absorptiometry for determining in vivo body composition of mice. Obes Res 8, 392-398.
Nelson K, Walsh D, Deeter P, Sheehan F (1994). A Phase II study of delta-9-tetrahydrocannabinol for appetite stimulation in cancer-associated anorexia. J Palliat Care; 10:14-8.
Plasse T F, Gorter R W, Krasnow S H, Lane M, Shepard K V, Wadleigh R G. (1991). Recent clinical experience with dronabinol. Pharmacol Biochem Behav; 40:695-670.
Popiela, T., Lucchi, R., Giongo, F. (1989). Methylprednisolone as palliative therapy for female terminal cancer patients. Eur J Cancer Clin Oncol; 25:1823-9.
Preston T, Fearon K C, McMillan D C, Winstanley F P, Slater C, Shenkin A, Carter D C. (1995). Effect of ibuprofen on the acute phase response and protein metabolism in patients with cancer and weight loss. Br J Surg; 82:229-34.
Raisanen, S. R., Alatalo, S. L., Ylipahkala, H., Halleen, J. M., Cassady, A. I., Hume, D. A., and Vaananen, H. K. (2005). Macrophages overexpressing tartrate-resistant acid phosphatase show altered profile of free radical production and enhanced capacity of bacterial killing. Biochem Biophys Res Commun 331, 120-126.
Reinholt, F. P., Widholm, S. M., Ek-Rylander, B., and Andersson, G. (1990). Ultrastructural localization of a tartrate-resistant acid ATPase in bone. J Bone Miner Res 5, 1055-1061.
Reynisdottir, S., Wahrenberg, H., Carlstrom, K., Rossner, S., and Amer, P. (1994). Catecholamine resistance in fat cells of women with pperbody obesity due to decreased expression of beta 2-adrenoceptors. Diabetologia 37, 428-435.
Ryden, M., Dicker, A., Van Harmelen, V., Hauner, H., Brunnberg, M., Perbeck, L., Lonnqvist, F., and Amer, P. (2002). Mapping of early singaling events in tumor necrosis factor-alpha-mediated lipolysis in human fat cells. J Biol Chem 277, 1085-1091.
Salans, L. B., Cushman, S. W., and Weismann, R. E. (1973). Studies of human adipose tissue. Adipose cell size and number in nonobese and obese patients. J Clin Invest 52, 929-941.
Salans, L. B., Knittle, J. L., and Hirsch, J. (1968). The role of adipose cell size and adipose tissue insulin sensitivity in the carbohydrate intolerance of human obesity. J Clin Invest 47, 153-165.
Shepherd, P. R., Gnudi, L., Tozzo, E., Yang, H., Leach, F., and Kahn, B. B. (1993). Adipose cell hyperplasia and enhanced glucose disposal in transgenic mice overexpressing GLUT4 selectively in adipose tissue. J Biol Chem 268, 22243-22246.
Shivshanker K, Bennett R W Jr, Haynie T P. (1983) Tumor-associated gastroparesis: correction with metoclopramide. Am J Surg; 145:221-5.
Simons J P, Aaronson N K, Vansteenkiste J F, ten Velde G P, Muller M J, Drenth B M, Erdkamp F L, Cobben E G, Schoon E J, Smeets J B, Schouten H C, Demedts M, Hillen H F, Blijham G H, Wouters E F. (1996). Effects of medroxyprogesterone acetate on appetite, weight and quality of life in advanced-stage non-hormone-sensitive cancer: a placebo-controlled multicenter study. J Clin Oncol; 14:1077-84.
Stern, J. S., Batchelor, B. R., Hollander, N., Cohn, C. K., and Hirsch, J. (1972). Adipose-cell size and immunoreactive insulin levels in obese and normal-weight adults. Lancet 2, 948-951.
Stumvoll, M., and Haring, H. U. (2002). Glitazones: clinical effects and molecular mechanisms. Ann Med 34, 217-24.
Tisdale M J, Dhesi J K. (1990) Inhibition of weight loss by omega-3 fatty acids in an experimental cachexia model. Cancer Res; 50:5022-6.
Tschop M, Statnick M A, Suter T M, Heiman M L. (2002). GH-releasing peptide-2 increases fat mass in mice lacking NPY: indication for a crucial mediating role of hypothalamic agoutirelated protein. Endocrinology; 143:558-68.
Uysal, K. T., Wiesbrock, S. M., Marino, M. W., and Hotamisligil, G. S. (1997). Protection from obesity-induced insulin resistance in mice lacking TNF-alpha function. Nature 389, 610-614.
Wahrenberg, H., Hertel, K., Leijonhufvud, B. M., Persson, L. G., Toft, E., and Amer, P. (2005). Use of waist circumference to predict insulin resistance: retrospective study. Bmj 330, 1363-1364.
Valet, P., Senard, J. M., Devedjian, J. C., Planat, V., Salomon, R., Voisin, T., Drean, G., Couvineau, A., Daviaud, D., Denis, C., and et al. (1993). Characterization and distribution of alpha 2-adrenergic receptors in the human intestinal mucosa. J Clin Invest 91, 2049-2057.
Walsh, N. C., Cahill, M., Carninci, P., Kawai, J., Okazaki, Y., Hayashizaki, Y., Hume, D. A., and Cassady, A. I. (2003). Multiple tissue-specific promoters control expression of the murine tartrate-resistant acid phosphatase gene. Gene 307, 111-123.
Wang, Y., Norgard, M., and Andersson, G. (2005). N-glycosylation influences the latency and catalytic properties of mammalian purple acid phosphatase. Arch Biochem Biophys 435, 147-156.
Weisberg, S. P., McCann, D., Desai, M., Rosenbaum, M., Leibel, R. L., and Ferrante, A. W., Jr. (2003). Obesity is associated with macrophage accumulation in adipose tissue. J Clin Invest 112, 1796-1808.
Wellen, K. E., and Hotamisligil, G. S. (2003). Obesity-induced inflammatory changes in adipose tissue. J Clin Invest 112, 1785-1788.
Weyer, C., Foley, J. E., Bogardus, C., Tataranni, P. A., and Pratley, R. E. (2000). Enlarged subcutaneous abdominal adipocyte size, but not obesity itself, predicts type II diabetes independent of insulin resistance. Diabetologia 43, 1498-1506.
Wigmore S J, Falconer J S, Plester C E, Ross J A, Maingay J P, Carter D C, Fearon K C. (1995). Ibuprofen reduces energy expenditure and acute-phase protein production compared with placebo in pancreatic cancer patients. Br J Cancer; 72:185-8.
Willox J C, Corr J, Shaw J, Richardson M, Calman K C, Drennan M. (1984). Prednisolone as an appetite stimulant in patients with cancer. Br Med J (Clin Res Ed); 288:27.
Winkler, G., Kiss, S., Keszthelyi, L., Sapi, Z., Ory, I., Salamon, F., Kovacs, M., Vargha, P., Szekeres, O., Speer, G., et al. (2003). Expression of tumor necrosis factor (TNF)-alpha protein in the subcutaneous and visceral adipose tissue in correlation with adipocyte cell volume, serum TNF-alpha, soluble serum TNF-receptor-2 concentrations and C-peptide level. Eur J Endocrinol 149, 129-135.
Väärniemi, J., Halleen, J. M., Kaarlonen, K., Ylipahkala, H., Alatalo, S. L., Andersson, G., Kaija, H., Vihko, P., and Väänänen, H. K. (2004). Intracellular machinery for matrix degradation in bone resorbing osteoclasts. J Bone Miner Res 19, 1932-1940.
Xu, H., Barnes, G. T., Yang, Q., Tan, G., Yang, D., Chou, C. J., Sole, J., Nichols, A., Ross, J. S., Tartaglia, L. A., and Chen, H. (2003). Chronic inflammation in fat plays a crucial role in the development of obesity-related insulin resistance. J Clin Invest 112, 1821-1830.

### Patent Documents

U.S. Pat. No. 6,274,336
U.S. Pat. No. 6,387,883
U.S. Pat. No. 7,012,075,
U.S. Pat. No. 7,015,241
U.S. Pat. No. 7,138,372
US patent application No. 20060122268.
WO04032952A1
WO04041170A2

1-24. (canceled)

25. A method of treatment of a mammal to increase the body fat mass of said mammal, or to prevent or reduce the loss of body fat mass of said mammal, comprising administering to a mammal in need an effective amount of a medicament comprising a polypeptide comprising an amino acid sequence having a sequence identity of at least 80%, with the amino acid sequence represented by Gly Asn Ser Asp Asp Phe Leu Ser Gln Gln Pro Glu Arg Pro Arg AspVal Lys Leu Ala Arg (SEQ ID NO: 2), or a pharmaceutically acceptable salt thereof.

26. The method according to claim 25, wherein said polypeptide comprises an amino acid sequence having a sequence identity of at least 80%, with the amino acid sequence represented by
(SEQ ID NO: 1)
Met Asp Met Trp Thr Ala Leu Leu Ile Leu Gln Ala
Leu Leu Leu Pro Ser Leu Ala Asp Gly Ala Thr Pro
Ala Leu Arg Phe Val Ala Val Gly Asp Trp Gly Gly
Val Pro Asn Ala Pro Phe His Thr Ala Arg Glu Met
Ala Asn Ala Lys Glu Ile Ala Arg Thr Val Gln Ile
Leu Gly Ala Asp Phe Ile Leu Ser Leu Gly Asp Asn
Phe Tyr Phe Thr Gly Val GIn Asp Ile Asn Asp Lys
Arg Phe Gln Glu Thr Phe Glu Asp Val Phe Ser Asp
Arg Ser Leu Arg Lys Val Pro Trp Tyr Val Leu Ala
Gly Asn His Asp His Leu Gly Asn Val Ser Ala Gln
Ile Ala Tyr Ser Lys Ile Ser Lys Arg Trp Asn Phe
Pro Ser Pro Phe Tyr Arg Leu His Phe Lys Ile Pro
Gln Thr Asn Val Ser Val Ala Ile Phe Met Leu Asp
Thr Val Thr Leu Cys Gly Asn Ser Asp Asp Phe Leu
Ser Gln Gln Pro Glu Arg Pro Arg Asp Val Lys Leu
Ala Arg Thr Gln Leu Ser Trp Leu Lys Lys Gln Leu
Ala Ala Ala Arg Glu Asp Tyr Val Leu Val Ala Gly
His Tyr Pro Val Trp 5cr Ile Ala Glu His Gly Pro
Thr His Cys Leu Val Lys Gln Leu Arg Pro Leu Leu
Ala Thr Tyr Gly Val Thr Ala Tyr Leu Cys Gly His
Asp His Asn Leu Gln Tyr Leu Gln Asp Glu Asn Gly
Val Gly Tyr Val Leu 5cr Gly Ala Gly Asn Phe Met
Asp Pro Ser Lys Arg His Gln Arg Lys Val Pro Asn
Gly Tyr Leu Arg Phe His Tyr Gly Thr Glu Asp Ser
Leu Gly Gly Phe Ala Tyr Val Glu Ile Ser Ser Lys
Glu Met Thr Val Thr Tyr Ile Glu Ala Ser Gly Lys
Ser Leu Phe Lys Thr Arg Leu Pro Arg Arg Ala Arg
Pro.

27. The method according to claim 26, wherein the polypeptide is the monomeric pro-enzyme of tartrate-resistant acid phosphatase type 5 (TRAP).

28. The method according to claim 25, wherein the medicament further contains at least one other biologically active substance.

29. The method according to claim 28, wherein the at least one other biologically active substance is selected from the group consisting of ghrelin, compounds capable of reducing the proteolytic processing of monomeric pro-enzyme TRAP, appetite stimulants and other substances which stimulate adipogenesis.

30. The method according to claim 25, wherein the mammal is one suffering from a condition associated with a subnormal body fat mass.

31. The method according to claim 25, wherein the mammal is one suffering from or susceptible of developing a condition selected from cachexia and anorexia nervosa.

32. A pharmaceutical composition comprising a polypeptide comprising an amino acid sequence having a sequence identity of at least 80%, with the amino acid sequence represented by Gly Asn Ser Asp Asp Phe Leu Ser Gln Gln Pro Glu Arg Pro Arg AspVal Lys Leu Ala Arg (SEQ ID NO: 2), or a pharmaceutically acceptable salt thereof, and a pharmaceutically acceptable carrier and or excipient.

33. The pharmaceutical composition according to claim 32, wherein the polypeptide comprises an amino acid sequence having a sequence identity of at least 80%, with the amino acid sequence represented by SEQ ID NO: 1.

34. The pharmaceutical composition according to claim 33, wherein the polypeptide is the monomeric pro-enzyme of TRAP.

35. The pharmaceutical composition according to claim 32, comprising at least one other biologically active substance.

36. The pharmaceutical composition according to claim 35, wherein the at least one other biologically active substance is selected from the group consisting of ghrelin, ghrelin analogs, compounds capable of reducing the proteolytic processing of monomeric pro-enzyme TRAP and appetite stimulants.

37. A method of treatment of a mammal to increase the body fat mass of said mammal, or to prevent or reduce the loss of body fat mass of said mammal, comprising administering to a mammal in need an effective amount of the pharmaceutical composition according to claim 32.

38. The method according to claim 25, wherein said sequence identity is at least 90%.

39. The method according to claim 25, wherein said sequence identity is at least 95%.

40. The method according to claim 26, wherein said sequence identity is at least 95%.

41. The method according to claim 26, wherein said sequence identity is at least 98%.

42. The pharmaceutical composition according to claim 32, wherein said sequence identity is at least 90%.

43. The pharmaceutical composition according to claim 32, wherein said sequence identity is at least 95%.

44. The pharmaceutical composition according to claim 33, wherein said sequence identity is at least 98%.

45. The method according to claim 29, wherein said other substances which stimulate adipogenesis is glitazones (thiazolidinediones).


SEQUENCE LISTING (13 sequences)

SEQ ID NO: 1 - protein, 325 aa, Homo sapiens
MDMWTALLILQALLLPSLADGATPALRFVAVGDWGGVPNAPFHTAREMANAKEIARTVQILGADFILSLGDNFYFTGVQDINDKRFQETFEDVFSDRSLRKVPWYVLAGNHDHLGNVSAQIAYSKISKRWNFPSPFYRLHFKIPQTNVSVAIFMLDTVTLCGNSDDFLSQQPERPRDVKLARTQLSWLKKQLAAAREDYVLVAGHYPVWSIAEHGPTHCLVKQLRPLLATYGVTAYLCGHDHNLQYLQDENGVGYVLSGAGNFMDPSKRHQRKVPNGYLRFHYGTEDSLGGFAYVEISSKEMTVTYIEASGKSLFKTRLPRRARP

SEQ ID NO: 2 - protein, 21 aa, Homo sapiens
GNSDDFLSQQPERPRDVKLAR

SEQ ID NO: 3 - DNA, 23 bp, Artificial
  Mouse TRAP sense primer sequence
gctacttgcggtttcactatgga

SEQ ID NO: 4 - DNA, 24 bp, Artificial
  Mouse TRAP antisense primer sequence
tggtcatttctttggggcttatct

SEQ ID NO: 5 - DNA, 24 bp, Artificial
  Mouse TRAP FAM labeled probe
tgtgaagccgcccagggagtcctc

SEQ ID NO: 6 - DNA, 19 bp, Artificial
  Human TRAP sense primer sequence
cgcacaggtaggcagtgac

SEQ ID NO: 7 - DNA, 20 bp, Artificial
  Human TRAP antisense primer sequence
ctaccccgtgtggtccatag

SEQ ID NO: 8 - DNA, 21 bp, Artificial
  Mouse TRAP transcript sense primer sequence
ggtcaggagtgggagccatat

SEQ ID NO: 9 - DNA, 24 bp, Artificial
  Mouse TRAP transcript antisense primer sequence
aagagccttcaagtaagtggaaca

SEQ ID NO: 10 - DNA, 20 bp, Artificial
  Mouse TRAP transcript sense primer sequence
tccgcagctcagttgggtag

SEQ ID NO: 11 - DNA, 21 bp, Artificial
  Mouse TRAP transcript antisense primer sequence
gcccacagccacaaatctcag

SEQ ID NO: 12 - DNA, 21 bp, Artificial
  Mouse TRAP transcript sense primer sequence
ctctgaccacctgtgcttcct

SEQ ID NO: 13 - DNA, 21 bp, Artificial
  Mouse TRAP transcript antisense primer sequence
ctgtgtggaatggggcattgg